(19) United States
(12) Patent Application Publication
(10) Pub. No.: US 2024/0420820 A1
(43) Pub. Date: Dec. 19, 2024

(54) INTEGRATING HEALTH DATA FROM MULTIPLE WEARABLE DEVICES

(71) Applicant: Dexcom, Inc., San Diego, CA (US)

(72) Inventors: Izabel PETERSON, San Diego, CA (US); Sonya Ann FRANGOPOULOS, San Diego, CA (US); Afshan A. KLEINHANZL, San Diego, CA (US); Stacey Lynne FISCHER, San Diego, CA (US); James LO, San Diego, CA (US); Daniya Alisa Petal ULGEN, San Diego, CA (US); Douglas S. KANTER, San Diego, CA (US)

(21) Appl. No.: 18/740,381

(22) Filed: Jun. 11, 2024

Related U.S. Application Data
(60) Provisional application No. 63/508,460, filed on Jun. 15, 2023.

(51) Int. Cl. G16H 20/30; G06F 1/16

(52) U.S. Cl. CPC G16H 20/30; G06F 1/163

(57) ABSTRACT

In some embodiments, one general aspect includes a method of integrating health data from multiple wearables. The method is performed by a system associated with a user and includes receiving, from an analyte sensor system worn by the user, a first time series of analyte levels of the user. The method also includes receiving, from an activity monitor worn by the user, activity information that identifies an activity of a defined activity type. The method also includes correlating the analyte levels to the activity based on a time period of the activity. The method also includes generating, by the system, integrative activity data based on the correlation of the analyte levels to the activity.

### CROSS-REFERENCE TO RELATED APPLICATIONS

This application claims priority to and benefit of U.S. Provisional Patent Application No. 63/508,460, filed Jun. 15, 2023, which is assigned to the assignee hereof and hereby expressly incorporated by reference herein in its entirety as if fully set forth below and for all applicable purposes.

### TECHNICAL FIELD

The present disclosure relates generally to medical devices such as analyte sensors, and more particularly, but not by way of limitation, to systems, devices, and methods for integrating health data from multiple wearable devices.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to several health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Blood sugar concentration levels may be monitored with an analyte sensor, such as a continuous glucose monitor. A continuous glucose monitor may provide the wearer (patient) with information such as an estimated blood glucose level, a trend of estimated blood glucose levels, etc.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In some embodiments, one general aspect includes a system for integrating health data from multiple wearables. The system includes an analyte sensor system worn by a user and an activity monitor worn by the user. The system also includes a computer system configured to communicate with the analyte sensor system and the activity monitor. The computer system is operable to receive, from the analyte sensor system worn by the user, a first time series of analyte levels of the user. The computer system is further operable to receive, from the activity monitor worn by the user, activity information that identifies an activity of a defined activity type. The computer system is further operable to correlate the analyte levels to the activity based on a time period of the activity. The computer system is further operable to generate integrative activity data based on the correlation of the analyte levels to the activity.

In some embodiments, another general aspect includes a method of integrating health data from multiple wearables. The method is performed by a system associated with a user and includes receiving, from an analyte sensor system worn by the user, a first time series of analyte levels of the user. The method also includes receiving, from an activity monitor worn by the user, activity information that identifies an activity of a defined activity type. The method also includes correlating the analyte levels to the activity based on a time period of the activity. The method also includes generating, by the system, integrative activity data based on the correlation of the analyte levels to the activity.

In some embodiments, another general aspect includes a computer-program product. The computer-program product includes a non-transitory computer-usable medium having computer-readable program code embodied therein. The computer-readable program code is adapted to be executed to implement a method. The method includes receiving, from an analyte sensor system worn by the user, a first time series of analyte levels of the user. The method also includes receiving, from an activity monitor worn by the user, activity information that identifies an activity of a defined activity type. The method also includes correlating the analyte levels to the activity based on a time period of the activity. The method also includes generating, by the system, integrative activity data based on the correlation of the analyte levels to the activity.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates an example of a health monitoring and support system, in accordance with certain embodiments.

FIG. 1B illustrates an example analyte sensor system including an example continuous analyte sensor(s) with sensor electronics, in accordance with certain embodiments.

FIG. 2 illustrates example inputs and example metrics that are generated based on the inputs, in accordance with certain embodiments.

FIG. 3 illustrates an example of a system for integrating health data from multiple wearable devices, in accordance with certain embodiments.

FIG. 4 illustrates example user interfaces for configuring a centralized health application to receive time-series data from an activity monitor, in accordance with certain embodiments.

FIG. 5 illustrates example user interfaces for synchronizing an activity monitor with a centralized health application, in accordance with certain embodiments.

FIG. 6 illustrates an example of a process for integrating health data from multiple wearable devices, in accordance with certain embodiments.

FIG. 7A illustrates an example of a user interface view that presents integrative activity data for a recorded activity, in accordance with certain embodiments.

FIG. 7B illustrates an example of a user interface view that presents additional integrative activity data for the recorded activity discussed relative to FIG. 7A, in accordance with certain embodiments.

FIG. 7C illustrates an example of a user interface view that presents an integrative activity insight for the recorded activity discussed relative to FIG. 7A, in accordance with certain embodiments.

FIG. 7D illustrates an example of a user interface view that presents an additional integrative activity insight for the recorded activity discussed relative to FIG. 7A, in accordance with certain embodiments.

FIG. 8A illustrates an example of a user interface view that is a glanceable activity card for a run, in accordance with certain embodiments.

FIG. 8B illustrates an example of a user interface view that is a glanceable activity card for a walk, in accordance with certain embodiments.

FIG. 8C illustrates an example of a user interface view that is a glanceable activity card for weightlifting, in accordance with certain embodiments.

FIG. 9 illustrates an example of a user interface view that integrated data for a recorded activity, in accordance with certain embodiments.

FIG. 10 illustrates an example of a user interface view that includes activity cards for a selectable groups of activities, in accordance with certain embodiments.

FIG. 11A illustrates an example of a user interface view that presents an integrative health-management insight, in accordance with certain embodiments.

FIG. 11B illustrates another example of a user interface view that presents an integrative health-management insight, in accordance with certain embodiments.

FIG. 12 illustrates an example of user interface views that present integrative activity data for a configurable period, in accordance with certain embodiments.

FIG. 13 illustrates another example of a user interface view that presents integrative activity data for a configurable period, in accordance with certain embodiments.

FIG. 14 illustrates an example of a user interface view that presents integrative activity data for a recorded activity, in accordance with certain embodiments.

FIG. 15 illustrates an example of a user interface view that presents integrative activity data with multiple event streams, in accordance with certain embodiments.

FIG. 16 illustrates another example of a user interface view that presents integrative activity data with multiple event streams, in accordance with certain embodiments.

FIG. 17 illustrates an example of a user interface view that presents integrative activity data for overlapping activities, in accordance with certain embodiments.

FIG. 18 illustrates another example of a user interface view that presents integrative activity data for overlapping activities, in accordance with certain embodiments.

FIG. 19 illustrates another example of a user interface view that presents integrative activity data for overlapping activities, in accordance with certain embodiments.

FIG. 20 is a block diagram depicting a computer system configured for integrating health data from multiple wearables, in accordance with certain embodiments.

### DETAILED DESCRIPTION

Management of diabetes can present complex challenges for patients, clinicians, and caregivers, as a confluence of many factors can impact a patient's glucose level and glucose trends. To assist patients with better managing this condition, portable or wearable medical devices (e.g., sensors and other types of monitoring and diagnostic devices) as well as a variety of diabetes intervention software applications (hereinafter “applications”) have been developed by various providers.

Many diabetes patients use two or more wearable devices alongside each other to gather different types of health data in an effort to manage their diabetes and overall health. For example, blood sugar concentration levels may be monitored with an analyte sensor, such as a continuous glucose monitor (CGM). A CGM may provide the wearer (patient) with information such as an estimated blood glucose level, a trend of estimated blood glucose levels, etc. In addition, activities and related data may be tracked with an activity monitor, such as a smartwatch or other activity tracker. In these cases, it is typically a manual process to use information provided by the two or more wearable devices to manage diabetes and understand the potential causes of glucose variation, for example, because two or more applications are typically involved to obtain the information. Thus, it is not generally feasible for diabetes patients to easily correlate their information from the separate wearables.

The present disclosure describes examples of a centralized health application executing, for example, on a user device such as a smartphone, that assimilates information from multiple wearables. For example, in certain embodiments, a first wearable may be an analyte sensor system such as a CGM, and a second wearable may be an activity monitor such as a smartwatch. For illustrative purposes, examples involving two wearables will be periodically described below, such that the first wearable is a CGM and the second wearable is a smartwatch. However, it should be appreciated that the principles described herein are similarly applicable to other wearables, such as other types of analyte monitoring systems and other types of activity monitors. In addition, although certain examples below are described in relation to a diabetic patient, the embodiments herein are similarly applicable and useful for managing information generated by a number of wearable devices for monitoring any disease or condition for any type of user and/or patient.

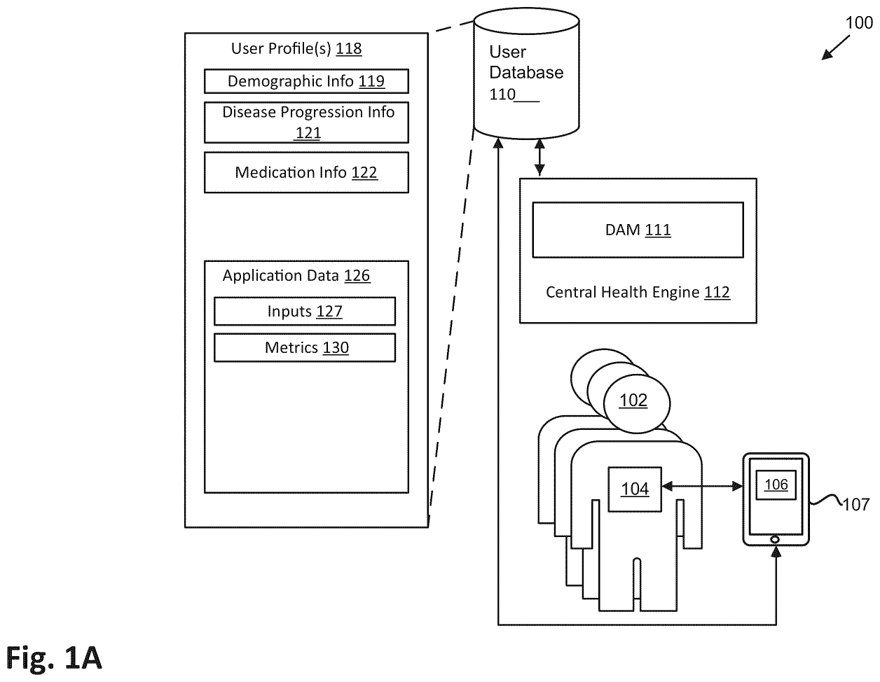

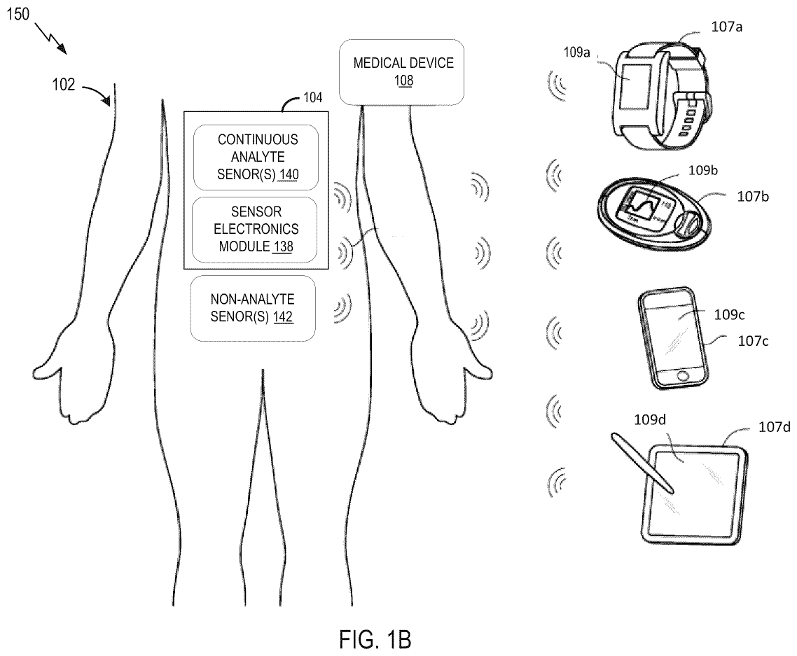

FIG. 1A illustrates an example of a health monitoring and support system 100, in accordance with certain embodiments of the disclosure. The health monitoring and support system 100 may be utilized for monitoring user health and displaying integrative data using various user interfaces to users associated with system 100.

Each user of system 100, such as user 102, may interact with a mobile health application, such as mobile health application (“application”) 106 (e.g., a diabetes intervention application that provides decision support guidance), and/or a health monitoring device, such as an analyte sensor system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte sensor system 104, a display device 107 that executes application 106, a central health engine 112, and a user database 110.

Analyte sensor system 104 may be configured to generate time-series data, such as analyte concentrations (e.g., sensor data), for the user 102, e.g., on a continuous basis, and transmit the analyte concentrations to the display device 107 for use by application 106. In some embodiments, the analyte sensor system 104 may transmit the analyte concentrations to the display device 107 through a wireless connection (e.g., Bluetooth connection). In certain embodiments, display device 107 is a smart phone. However, in certain embodiments, display device 107 may instead be any other type of computing device such as a laptop computer, a smartwatch, a tablet, or any other computing device capable of executing application 106.

Note that, while in certain examples the analyte sensor system 104 is assumed to be a glucose monitoring system, analyte sensor system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates).

Analytes can include naturally occurring substances, artificial substances, metabolites, and/or reaction products. In some embodiments, the analyte measured and used by the devices and methods described herein may include albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, ketones, sodium, total protein, uric acid, metabolic markers, and/or drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free j-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Application 106 may be a mobile health application that is configured to receive and analyze time-series data, including analyte concentrations, from the analyte sensor system 104 and/or other devices, as described in greater detail relative to FIGS. 1B, 2, and 3. In some embodiments, application 106 may transmit analyte concentrations received from the analyte sensor system 104 to a user database 110 (and/or the central health engine 112), and the user database 110 (and/or the central health engine 112) may store the analyte concentrations in a user profile 118 of user 102 for processing and analysis as well as for use by the central health engine 112 to generate integrative data based on an automatic integration of the analyte concentrations with data from other devices or sources. In various embodiments, application 106 may provide the integrative data to the user 102 using user interfaces via the application 106. In some embodiments, application 106 may store the analyte concentrations in a user profile 118 of user 102 locally for processing and analysis as well as for use by the central health engine 112 to determine and present integrative data using user interfaces to the user 102.

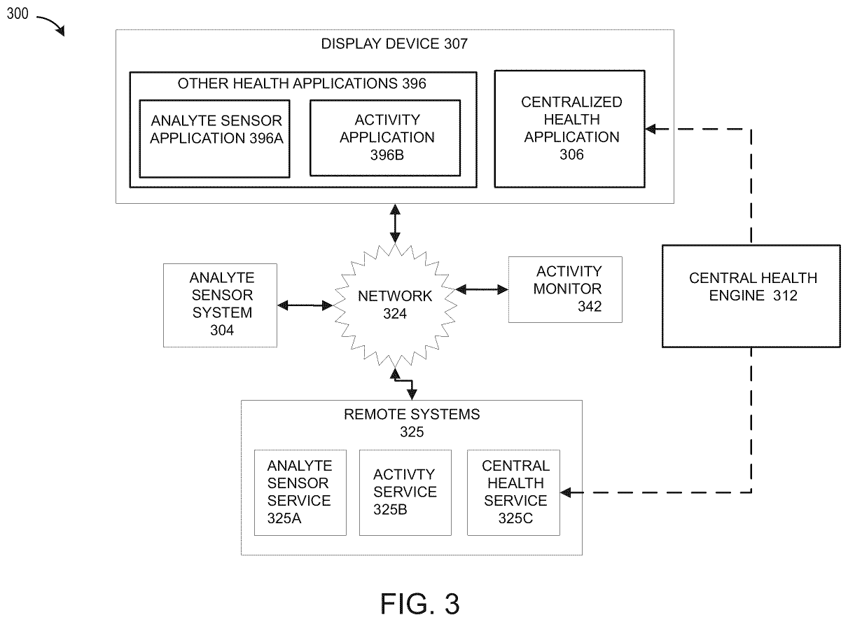

In certain embodiments, central health engine 112 refers to a set of software instructions with one or more software modules, including a data analysis module (DAM) 111. In some embodiments, central health engine 112 executes entirely on one or more computing devices in a private or a public cloud. In some other embodiments, central health engine 112 executes partially on one or more local devices, such as display device 107 (e.g., via application 106) and/or analyte sensor system 104, and partially on one or more computing devices in a private or a public cloud. In some other embodiments, central health engine 112 executes entirely on one or more local devices, such as display device 107 (e.g., via application 106) and/or analyte sensor system 104. As will be discussed in more detail relative to FIGS. 3-19, central health engine 112, may determine integrative data and present the integrative data to the user via the application 106.

In certain embodiments, DAM 111 of central health engine 112 may be configured to receive and/or process a set of inputs 127 (described in more detail below) (also referred to herein as “input data”) to determine one or more metrics 130 (also referred to herein as “metrics data”). Inputs 127 may be stored in the user profile 118 in the user database 110. DAM 111 can fetch inputs 127 from the user database 110 and compute a plurality of metrics 130 which can then be stored as application data 126 in the user profile 118. Such metrics 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the central health engine 112, over one or more networks (not shown).

In certain embodiments, application 106 collects inputs 127 through user 102 input and/or a plurality of other sources, including analyte sensor system 104, other applications running on display device 107, and/or one or more other sensors and devices. In certain embodiments, such sensors and devices include one or more of, but are not limited to, an insulin pump, other types of analyte sensors, sensors or devices provided by display device 107 (e.g., accelerometer, camera, global positioning system (GPS), heart rate monitor, etc.) or other user accessories (e.g., a smartwatch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of data store, such as relational databases, non-relational databases, key-value data stores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by health monitoring and support system 100. More particularly, similar to the operations performed with respect to the user 102, the operations performed with respect to these other users may utilize an analyte monitoring system, such as analyte sensor system 104, and also interact with the same application 106, copies of which execute on the respective display devices of the other users 102. For such users, user profiles 118 are similarly created and stored in user database 110.

FIG. 1B is a diagram 150 illustrating an example analyte sensor system 104 including an example continuous analyte sensor(s) with sensor electronics, in accordance with certain aspects of the present disclosure. For example, the analyte sensor system 104 may be configured to continuously monitor one or more analytes of a user, in accordance with certain aspects of the present disclosure.

As shown in FIG. 1B, the analyte sensor system 104 in the illustrated embodiment includes a sensor electronics module 138 and one or more continuous analyte sensor(s) 140 (individually referred to herein as the continuous analyte sensor 140 and collectively referred to herein as the continuous analyte sensors 140) associated with a sensor electronics module 138. The sensor electronics module 138 may be in wireless communication (e.g., directly or indirectly) with one or more display devices 107a, 107b, 107c, and 107d. In certain embodiments, the sensor electronics module 138 may also be in wireless communication (e.g., directly or indirectly) with one or more medical devices 108 (individually referred to herein as the medical device 108 and collectively referred to herein as the medical devices 108).

In certain embodiments, a continuous analyte sensor 140 may comprise a sensor for detecting and/or measuring analyte(s). The continuous analyte sensor 140 may be a multi-analyte sensor configured to continuously measure two or more analytes (e.g., ketone, glucose) or a single analyte sensor configured to continuously measure a single analyte as a non-invasive device, a subcutaneous device, a transcutaneous device, a transdermal device, and/or an intravascular device.

In certain embodiments, the continuous analyte sensor 140 may be configured to continuously measure analyte levels of a user using one or more measurement techniques, such as enzymatic, chemical, physical, electrochemical, spectrophotometric, polarimetric, calorimetric, iontophoretic, radiometric, immunochemical, and the like. In certain aspects, the continuous analyte sensor 140 provides a data stream indicative of the concentration of one or more analytes in the user. The data stream may include raw data signals which may be converted into a calibrated and/or filtered data stream used to provide estimated analyte value(s) to the user.

In certain embodiments, the continuous analyte sensor 140 may be a multi-analyte sensor, configured to continuously measure multiple analytes in a user's body. For example, in certain embodiments, the continuous multi-analyte sensor 140 may be a single sensor configured to measure glucose, ketones, and/or other blood analytes in the user's body.

In certain embodiments, one or more multi-analyte sensors may be used in combination with one or more single analyte sensors. As an illustrative example, a multi-analyte sensor may be configured to continuously measure ketone and glucose and may, in some cases, be used in combination with one or more other analyte sensors configured to measure only, for example, hydration levels or protein levels. Information from each of the multi-analyte sensor(s) and single analyte sensor(s) may be combined to provide integrative data using methods described herein.

In certain embodiments, the sensor electronics module 138 includes electronic circuitry associated with measuring and processing the continuous analyte sensor data, including prospective algorithms associated with processing and calibration of the sensor data. The sensor electronics module 138 can be physically connected to the continuous analyte sensor(s) 140 and can be integral with (non-releasably attached to) or releasably attachable to the continuous analyte sensor(s) 140. The sensor electronics module 138 may include hardware, firmware, and/or software that enables measurement of levels of analyte(s) via a continuous analyte sensor(s) 140. For example, the sensor electronics module 138 can include a potentiostat, a power source for providing power to the sensor, other components useful for signal processing and data storage, and a telemetry module for transmitting data from the sensor electronics module to one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

In some embodiments, the display devices 107a, 107b, 107c, and/or 107d are configured for displaying displayable sensor data, including analyte data, which may be transmitted by the sensor electronics module 138. In addition, or alternatively, the display devices 107a, 107b, 107c, and/or 107d are configured for displaying integrative data as described herein, which data can be generated by the central health engine 112. Each of the display devices 107a, 107b, 107c, or 107d can include a display such as a touchscreen display 109a, 109b, 109c,/or 109d for displaying sensor data to a user and/or receiving inputs from the user. For example, a graphical user interface may be presented to the user for such purposes. In some embodiments, the display devices 107a, 107b, 107c, and 107d may include other types of user interfaces such as a voice user interface instead of, or in addition to, a touchscreen display for communicating sensor data to the user of the display device and/or receiving user inputs. The display devices 107a, 107b, 107c, and 107d may be examples of the display device 107 illustrated in FIG. 1A used to display sensor data to the user 102 and/or receive input from the user 102.

In some embodiments, one, some, or all of the display devices are configured to display or otherwise communicate the sensor data as it is communicated from the sensor electronics module (e.g., in a data package that is transmitted to respective display devices), without any additional prospective processing required for calibration and real-time display of the sensor data.

The plurality of display devices may include a custom display device specially designed for displaying certain types of displayable sensor data associated with analyte data received from sensor electronics module and/or integrative data. In certain embodiments, the plurality of display devices may be configured for providing alerts/alarms based on the displayable sensor data. The display device 107b is an example of such a custom device. In some embodiments, one of the plurality of display devices is a smartphone, such as the display device 107c which represents a mobile phone, using a commercially available operating system (OS), and configured to display a graphical representation of the continuous sensor data (e.g., including current and historic data) and/or integrative data. Other display devices can include other hand-held devices, such as the display device 107d which represents a tablet, the display device 107a which represents a smartwatch, the medical device 108 (e.g., an insulin delivery device or a blood glucose meter), and/or a desktop or laptop computer (not shown). Display device 107d and display device 107a may similarly be configured to display graphical representations of the continuous sensor data (e.g., including current and historic data) and/or integrative data.

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 138 that is physically connected to continuous analyte sensor(s) 140) during a sensor session to enable a plurality of different types and/or levels of display and/or functionality associated with the displayable sensor data. In certain embodiments, the type of alarms customized for each particular display device, the number of alarms customized for each particular display device, the timing of alarms customized for each particular display device, and/or the threshold levels configured for each of the alarms (e.g., for triggering) are based on the current health of a user, the state of a user's analyte levels, current treatment recommended to a user, and/or physiological parameters of a user.

As mentioned, the sensor electronics module 138 may be in communication with a medical device 108. The medical device 108 may be a passive device in some example embodiments of the disclosure. For example, the medical device 108 may be an insulin pump for administering insulin to a user.

In certain embodiments, one or more other non-analyte sensors 142 (individually referred to herein as the non-analyte sensor 142 and collectively referred to herein as the non-analyte sensor 142) may be in communication with any of the display devices 107. The non-analyte sensors 142 may include, but are not limited to, an altimeter sensor, an accelerometer sensor, a temperature sensor, a respiration rate sensor, a sweat sensor, etc. The non-analyte sensors 142 may also include monitors such as heart rate monitors, ECG monitors, blood pressure monitors, pulse oximeters, or the like. The non-analyte sensors 142 may also include data systems for measuring non-patient specific phenomena such as time, ambient pressure, or ambient temperature which could include an atmospheric pressure sensor, an external air temperature sensor or a clock, timer. In some embodiments, the non-analyte sensors 142 may be, or include, an activity monitor, for example, that includes a combination of the foregoing sensors, such as an accelerometer sensor, a heart rate monitor, GPS sensor, and/or the like. In addition, or alternatively, the non-analyte sensors 142, such as an activity monitor, may be, or be integrated in, one or more of the display devices 107 such as, for example, the display device 107a which represents a smartwatch. One or more of these non-analyte sensors 142 may provide data to the central health engine 112.

In certain embodiments, a wireless access point (WAP) may be used to couple one or more of the analyte sensor system 104, the plurality of display devices, the medical device(s) 108, and/or the non-analyte sensor(s) 142 to one another. For example, the WAP may provide Wi-Fi and/or cellular connectivity among these devices. Near Field Communication (NFC) and/or Bluetooth may also be used among devices depicted in the diagram 150 of FIG. 1B.

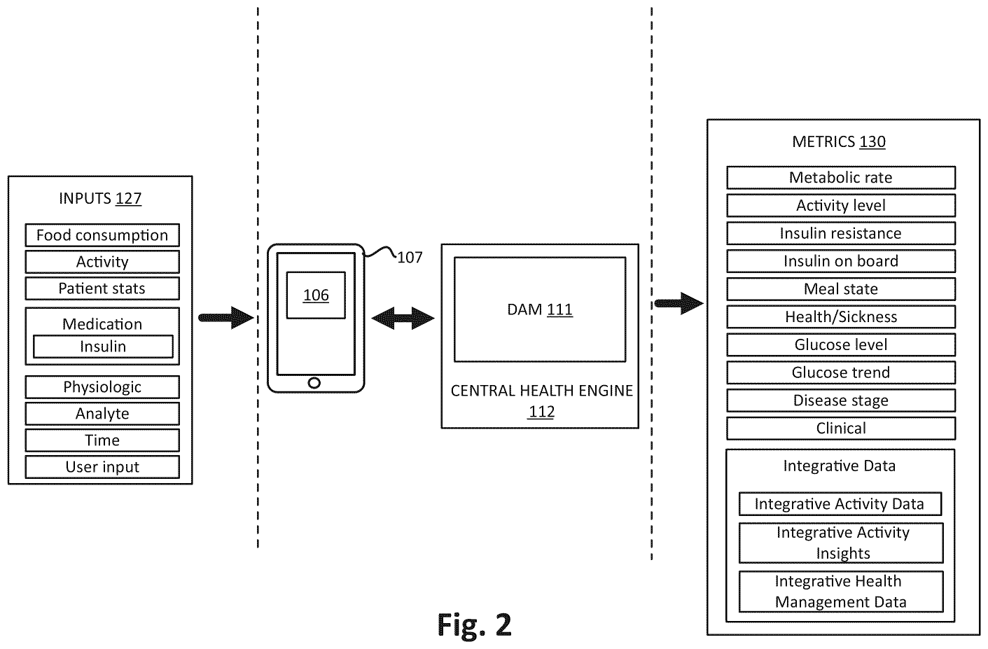

FIG. 2 illustrates example inputs and example metrics that are generated based on the inputs in accordance with certain embodiments of the disclosure. In particular, FIG. 2 illustrates example inputs 127 on the left, application 106 and central health engine 112, with DAM 111, in the middle, and example metrics 130 on the right. In certain embodiments, application 106 may obtain inputs 127, in the form of time-series data, through one or more channels (e.g., continuous analyte sensor(s) 104, non-analyte sensor(s) 142, various applications executing on display device 107, etc.). Inputs 127 may be further processed by DAM 111 to output a plurality of metrics, such as metrics 130. Further, inputs (e.g., inputs 127) and metrics (e.g., metrics 130) may be used by the DAM 111 and/or any computing device in the system 100 to perform various processes in determining and displaying integrative data to users, as further described below. Any of inputs 127 may be used for computing any of metrics 130. In certain embodiments, each one of metrics 130 may correspond to one or more values, e.g., discrete numerical values, ranges, or qualitative values (high/medium/low or stable/unstable). In some embodiments, some or all of metrics 130 may include time-series data and/or be provided in the form of time-series data.

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or context (e.g., workday breakfast at home, weekend brunch at restaurant). In some examples, meal information may be received via a convenient user interface provided by application 106.

In certain embodiments, inputs 127 include activity information. Activity information may be provided, for example, the one or more non-analyte sensors 142 of FIG. 1B. In certain embodiments, activity information may additionally be provided through manual input by user 102. Activity information may include, for example, a time series for each of heart rate, activity minutes, step count, floors climbed, location information (e.g., GPS data), calories burned, sleep duration and/or quality, activity level (e.g., light, medium, or heavy), and/or similar information. In addition, or alternatively, the activity information can include one or more time series for recorded activities of one or more defined activity types (e.g., walk, run, sprint, swim, weightlift etc.), where each activity is associated with a duration and/or time period.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the display device 107 to provide patient data.

In certain embodiments, inputs 127 include information relating to the user's medication intake. For example, the user's medication intake may include the user's insulin delivery. Such information may be received, via a wireless connection on a smart pen, via user input, and/or from an insulin pump (e.g., medical device 108). Insulin delivery information may include one or more of insulin volume, time of delivery, etc. Other configurations, such as insulin action time or duration of insulin action, may also be received as inputs.

In certain embodiments, inputs 127 include physiological information received from non-analyte sensor(s) 142, which may detect one or more of heart rate, respiration, oxygen saturation, body temperature, etc. (e.g., to detect illness, stress levels, etc.).

In certain embodiments, inputs 127 include analyte data, which may be provided as input from analyte sensor system 104, for example, in any of the ways described with respect to FIG. 1A. An example of analyte data is glucose data, which may be provided and/or stored as a time series corresponding to time-stamped glucose measurements over time. Other types of analyte data, such as ketone data, potassium data, lactate data, etc., may similarly be provided and/or stored as a time series.

In certain embodiments, inputs 127 include time, such as time of day, or time from a real-time clock.

As described above, in certain embodiments, DAM 111 determines or computes metrics 130 based on inputs 127 associated with user 102. An example list of metrics 130 is illustrated in FIG. 2. In certain embodiments, metrics 130 determined or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, metrics 130 determined or computed by DAM 111 include an activity level metric. The activity level metric may indicate a level of activity of the user. In certain embodiments, the activity level metric may be determined, for example based on input from an activity sensor or other physiologic sensors. In certain embodiments, the activity level metric may be calculated by DAM 111 based on one or more of inputs 127, such as one or more of activity information, physiological information, analyte data, time, user input, etc. Activity level may indicate whether the user is exercising, at rest, sleeping, etc.

In certain embodiments, metrics 130 determined or computed by DAM 111 include an insulin resistance metric (also referred to herein as an “insulin resistance”). The insulin resistance metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, metrics 130 determined or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

In certain embodiments, metrics 130 determined or computed by DAM 111 include health and sickness metrics. Health and sickness metrics may be determined, for example, based on one or more of user input (e.g., pregnancy information or known sickness information), from non-analyte sensor(s) 142, such as physiologic sensors (e.g., temperature), activity sensors, or a combination thereof. In certain embodiments, based on the values of the health and sickness metrics, for example, the user's state may be defined as being one or more of healthy, ill, rested, or exhausted. In certain embodiments, health and sickness metric may indicate the user's heart rate, stress level, etc.

In certain embodiments, metrics 130 determined or computed by DAM 111 include analyte level metrics. Analyte level metrics may be determined from analyte data (e.g., glucose measurements obtained from analyte sensor system 104). In some examples, an analyte level metric may also be determined, for example, based upon historical information about analyte levels in particular situations, e.g., given a combination of food consumption, insulin, and/or activity. An analyte level metric may include a rate of change of the analyte, time in range, time spent below a threshold level, time spent above a threshold level, or the like. In certain embodiments, an analyte trend may be determined based on the analyte level over a certain period of time. As described above, example analytes may include glucose, ketones, lactate, potassium and others described herein.

In certain embodiments, metrics 130 determined or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, metrics 130 determined or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from glucose values. In certain embodiments, clinical metrics may also include one or more of estimated A1C, glycemic variability, hypoglycemia, and/or health indicator (time magnitude out of target zone).

In certain embodiments, metrics 130 determined or computed by DAM 111 include integrative data that assimilates, for example, time-series data from multiple devices or wearables, such as time-series data from the analyte sensor system 104 and one or more of the non-analyte sensors 142. In some examples, the integrative data can include integrative activity data. The integrative activity data can include, or result from, a summarization, transformation, or analysis of activity information together with other information such as analyte information, medication information, and/or the like.

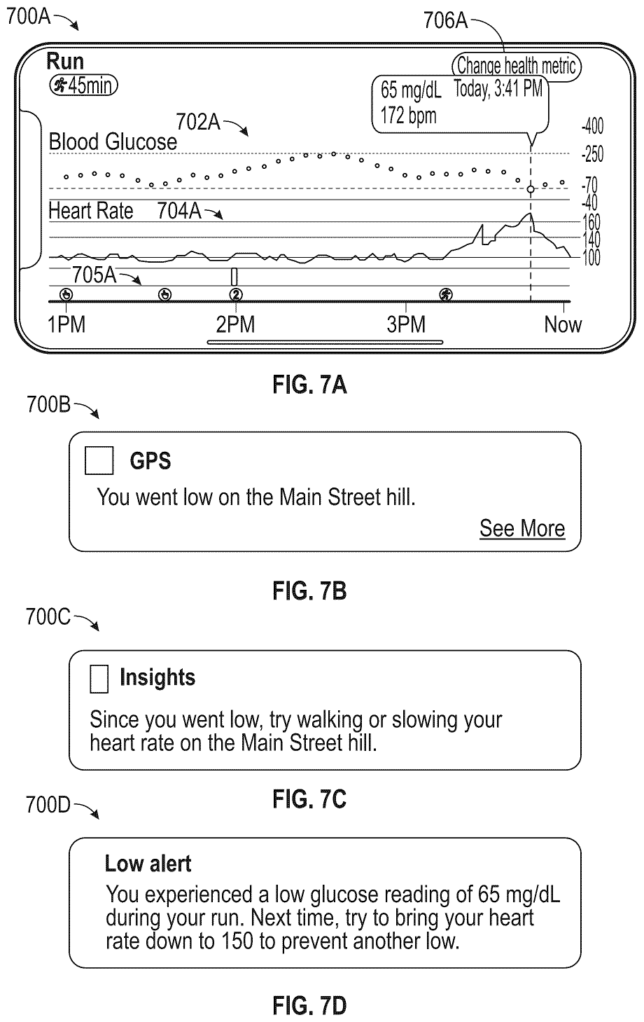

Continuing the foregoing examples, the integrative data can include integrative activity insights. The integrative activity insights can include, for example, recommendations or advice related to future activities or behaviors of the user, where the recommendations or advice is based on values and/or relationships between values in the integrative activity data. In addition, or alternatively, the integrative data can include integrative health-management insights. For example, in some embodiments, the integrative health-management insights can include activity recommendations for the user based on an automated analysis of the integrative activity data over a configurable period of time. In various embodiments, the dynamic determination and presentation of integrative data may utilize various user interfaces, as further described below. Various types of integrative data are illustrated in FIGS. 7A-D, 8A-C, and 9-19.

FIG. 3 illustrates an example of a system 300 for integrating health data from multiple wearables and/or other devices. The system 300 includes an analyte sensor system 304, a display device 307, an activity monitor 342, a network 324, a central health engine 312, and/or remote systems 325. In general, the network 324 can encompass any method for communication between two or more components connected thereto, inclusive of any of the communication methods or protocols described relative to FIGS. 1A-B and 2.

The analyte sensor system 304 can operate as described relative to the analyte sensor system 104 of FIG. 1. More generally, the analyte sensor system 304 is operable to acquire and/or generate time-series data related to health conditions or circumstances monitored thereby. The time-series data acquired or generated by the analyte sensor system 304 can include a plurality of time series inputs, such as time series corresponding to various of the inputs 127 of FIG. 2.

For example, the analyte sensor system 304 can acquire or generate a time series of analyte concentrations (e.g., glucose levels), where the analyte concentrations are indexed in time order, for example, at substantially equally spaced points in time (e.g., every second, every minute, every 5 minutes, every 10 minutes, etc.). In addition, or alternatively, in some embodiments, the analyte sensor system 304 can acquire or generate a time series of other information potentially stored by the analyte sensor system 304, such as medication information, food consumption information, or other information.

The activity monitor 342 can be a smartwatch or another device that operates, for example, as described relative to the non-analyte sensors 142 of FIG. 1B. More generally, the activity monitor 342 is operable to acquire or generate time-series data related to health conditions or circumstances monitored thereby. The time-series data acquired or generated by the activity monitor 342 can include multiple time series, such as a time series for each set of activity information discussed relative to the inputs 127 of FIG. 2.

For example, the activity monitor 342 can acquire or generate a time series for each of heart rate, activity minutes, step count, floors climbed, location information (e.g., GPS data), calories burned, sleep duration and/or quality, and/or similar physiological information. In addition, or alternatively, the activity monitor 342 can acquire or generate activity information that identifies one or more recorded activities of one or more defined activity types (e.g., walk, run, sprint, swim, weightlift etc.), where each activity is associated with a duration and/or time period. In various embodiments, the activity monitor can acquire or generate one or more time series for the recorded activities. In various embodiments, the recorded activities can be user-indicated and/or detected by the activity monitor 342. In some of these embodiments, a time series for an activity of a given type can be associated with other time series generated by the activity monitor 342 (e.g., heart rate or GPS) and/or other activity information such as intensity (e.g., light, medium, or heavy) and/or the like.

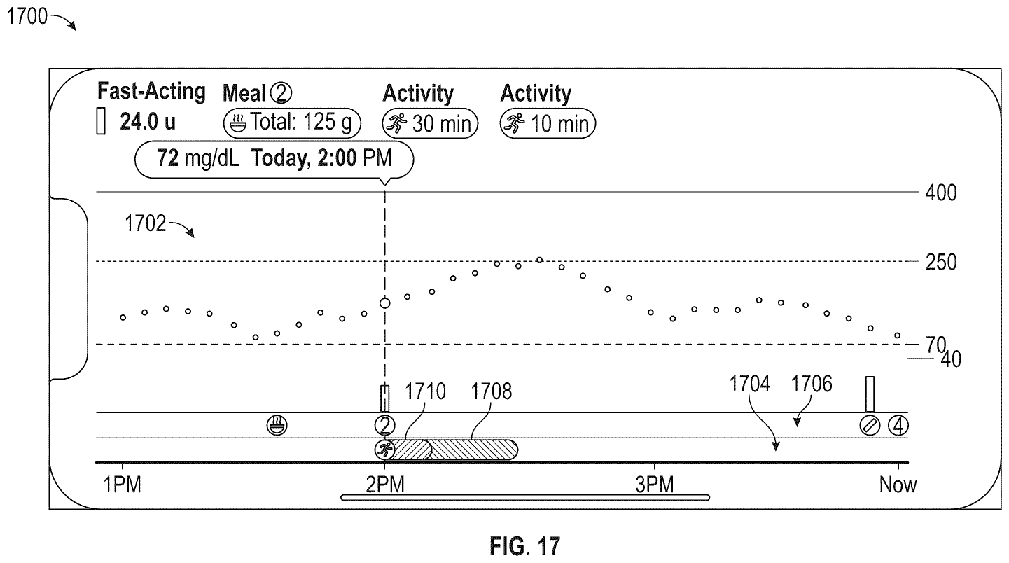

In certain embodiments, certain of the recorded activities can overlap, for example, such that at least a portion of one activity occurs during at least a portion of another activity (e.g., a sprint activity within a longer running activity). In some cases, the overlapping activities can share a start time or end time. Accordingly, in these embodiments, the one or more time series from the activity monitor 342 can identify the overlapping activities. Examples of overlapping activities will be described in greater detail relative to FIGS. 17-19.

The central health engine 312 may operate as described relative to the central health engine 112 of FIGS. 1A-B and 2. In the example of FIG. 3, functionality of the central health engine 112 is shown as being distributed between the centralized health application 306 and central health service 325C, further discussed below. For illustrative purposes, some features may be periodically described as being performed by the centralized health application 306 or the central health service 325C, while at other times functionality may be described as being performed by the central health engine 312 generally. It should be appreciated, however, that, unless specifically stated otherwise, or otherwise understood within the context described, any such features can be performed by any component to which the functionality of the central health engine 312 may be distributed, including, but not limited to, the centralized health application 306 and the central health service 325C.

The display device 307 may operate as described relative to any of the display devices 107 discussed relative to FIGS. 1A-B and 2. As illustrated in the example of FIG. 3, the display device 307 has a centralized health application 306 and other health applications 396 resident and executing thereon. The centralized health application 306 can operate, for example, as described relative to the application 106 of FIG. 1A. In various embodiments, the centralized health application 306 is operable to control the display device 307 to receive and integrate time-series data from the analyte sensor system 304, the activity monitor 342, and/or other devices or systems. The other health applications 396 can include, for example, an analyte sensor application 396A associated with managing or communicating with the analyte sensor system 304 and an activity application 396B associated with managing or communicating with the activity monitor 342.

In some embodiments, the centralized health application 306 can be the same application as one of the other health applications 396. For example, in some embodiments, the centralized health application 306 can be the same application as the analyte sensor application 396A, such that the analyte sensor application 396A is further operable to integrate time-series data from the activity monitor 342. In other embodiments, the centralized health application 306 can be the same application as the activity application 396B, such that the activity application 396B is further operable to integrate time-series data from the analyte sensor system 304. In still other embodiments, the centralized health application 306 can be independent of both the analyte sensor application 396A and the activity application 396B. Other examples will be apparent to one skilled in the art after a detailed review of the present disclosure.

The remote systems 325 can include external systems with which the analyte sensor system 304, the activity monitor 342, and/or the display device 307 may interact to perform their functionality. In some embodiments, the remote systems 325 may be deployed in public or private cloud environments. For illustrative purposes, the remote systems 325 are shown to include an analyte sensor service 325A, an activity service 325B, and a central health service 325C. It should be appreciated that the remote systems 325 can be greater or fewer in number to suit a given implementation. For example, in some embodiments, the central health service 325C can be, or correspond to, the analyte sensor service 325A, such that the analyte sensor service 325A performs functionality described relative to the analyte sensor service 325A and functionality described relative to the central health service 325C. In another example, the central health service 325C can be, or correspond to, the activity service 325B, such that the activity service 325B performs functionality described relative to the activity service 325B and functionality described relative to the central health service 325C. In yet another example, the central health service 325C can perform functionality described relative to the analyte sensor service 325A, the activity service 325B, and the central health service 325C.

In various embodiments, certain operations discussed herein relative to the analyte sensor system 304 or the activity monitor 342 can be allocated or distributed, at least in part, to the display device 307 (e.g., the other health applications 396) and/or the remote systems 325. For example, the analyte sensor system 304 and the activity monitor 342, by virtue of their respective provider or manufacturer, may be associated with the analyte sensor application 396A and the activity application 396B, respectively, and/or with the analyte sensor service 325A and activity service 325B, respectively. Although multiple systems or devices may be involved in acquiring, generating, storing, or providing data based on the operations of the analyte sensor system 304 and the activity monitor 342, for ease of description, such data may be described herein as being acquired, generated, stored, or provided by the analyte sensor system 304 and the activity monitor 342, respectively.

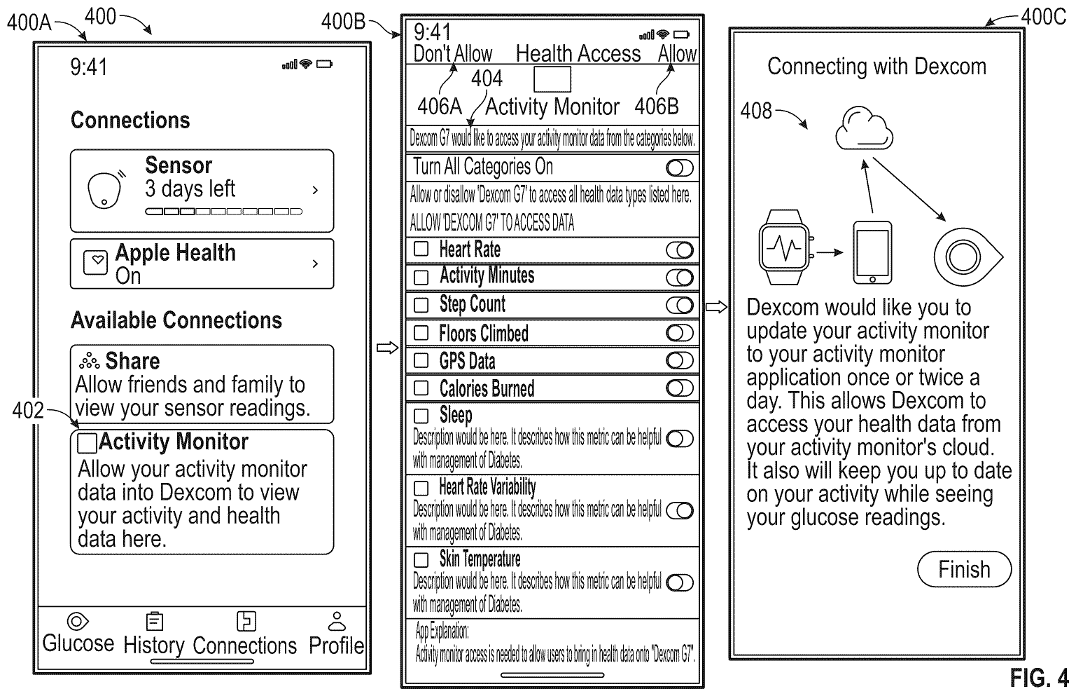

In various embodiments, the centralized health application 306 is operable to integrate health data from multiple wearables, such as the analyte sensor system 304 and the activity monitor 342. In some embodiments, the centralized health application 306 can guide the user through a procedure for configuring the centralized health application 306 to receive time-series information generated by one or both of the analyte sensor system 304 and the activity monitor 342. An example procedure for configuring the centralized health application 306 to receive time-series information generated by the activity monitor 342 will be described relative to FIG. 4.

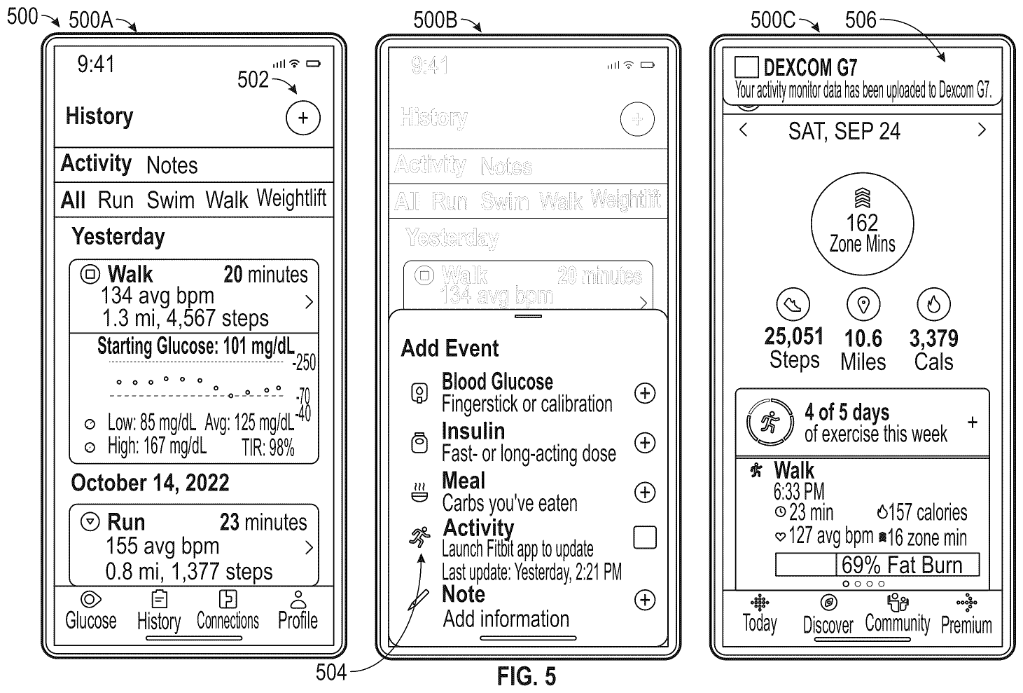

In some embodiments, the centralized health application 306 can receive time-series data from the analyte sensor system 304 and the activity monitor 342 on a continuous basis. In certain other embodiments, the centralized health application 306 can receive time-series data from the analyte sensor system 304 and/or the activity monitor 342 non-continuously, for example, in response to user-initiated synchronization. For example, in some embodiments, the analyte sensor system 304 may provide analyte concentrations and/or other time-series data on a continuous basis as described previously, while the activity monitor 342 may provide time-series data, such as activity information, in response to user-initiated synchronization of the activity monitor 342 with the centralized health application 306 and/or the activity application 396B. An example of synchronization will be described relative to FIG. 5.

FIG. 4 illustrates examples of user interfaces 400 for configuring the centralized health application 306 to receive time-series data from the activity monitor 342 via user-initiated synchronization. The user interfaces 400 can be presented, for example, on the display device 307. In the example of FIG. 4, the centralized health application 306 corresponds to the same application as the analyte sensor application 396A. The user interfaces 400 include a user interface 400A, a user interface 400B, and a user interface 400C.

In various embodiments, the configuration of the display device 307 to receive time-series data from the activity monitor 342 can begin with the display device 307 presenting the user interface 400A. As illustrated, the user interface 400A provides a user-selectable option 402 for initiating the configuration. If, for example, the centralized health application 306 receives a user selection of the user-selectable option 402, the centralized health application 306 may cause the user interface 400B to be presented on the display device 307.

In the example of FIG. 4, the user interface 400B enables user editing of access settings 404 for individual data parameters of the activity monitor 342. In various embodiments, the user interface 400B may be an interface provided by the centralized health application 306, the activity application 396B, an operating system of the display device 307, and/or the like. The user interface 400B further includes a denial option 406A and an allowance option 406B. User selection of the denial option 406A may result in termination of the configuration. If the display device 307 receives a user selection of the allowance option 406B, the centralized health application 306 may be given access to the data parameters of the activity monitor 342 in accordance with the access settings 404, in which case the centralized health application 306 causes the user interface 400C to be presented on the display device 307.

Continuing the example of FIG. 4, the user interface 400C provides a prompt 408 instructing the user to periodically open the activity application 396B, for example, for purposes of synchronizing the activity monitor 342 therewith. In various embodiments, each synchronization of the activity monitor 342 with the activity application 396B can result in time-series data from the activity monitor 342 being stored in a location accessible to the centralized health application 306, such as on the display device 307 and/or in the activity service 325B, thereby further enabling synchronization with the centralized health application 306.

FIG. 5 illustrates examples of user interfaces 500 for synchronizing the activity monitor 342 with the centralized health application 306. The user interfaces 500 can be presented, for example, on the display device 307. In the example of FIG. 5, the centralized health application 306 corresponds to the same application as the analyte sensor application 396A. The user interfaces 500 include a user interface 500A, a user interface 500B, and a user interface 500C.

In various embodiments, the synchronization of the activity monitor 342 with the centralized health application 306 can begin with the display device 307 presenting the user interface 500A. As illustrated, the user interface 500A provides a user-selectable option 502 for adding configurable types of information as user-inputted events. If, for example, the centralized health application 306 receives a user selection of the user-selectable option 502, the centralized health application 306 may cause the user interface 500B to be presented on the display device 307.

In the example of FIG. 5, the user interface 500B includes a user-selectable option 504 to launch the activity application 396B, for example, for purposes of synchronizing the activity monitor 342 therewith (shown as a user-inputted event in FIG. 5). If, for example, the centralized health application 306 receives a user selection of the user-selectable option 504, the centralized health application 306 may launch the activity application 396B, thereby causing the user interface 500C to be presented on the display device 307.

Continuing with the example of FIG. 5, the user interface 500C may be provided, for example, by the activity application 396B, as the activity application 396B synchronizes with the activity monitor 342. In various embodiments, the synchronization of the activity monitor 342 with the activity application 396B can result in time-series data from the activity monitor 342 being stored in a location accessible to the centralized health application 306, such as on the display device 307 and/or in the activity service 325B, thereby further enabling synchronization with the centralized health application 306. In various embodiments, the centralized health application 306 can receive and store the time-series data, thereby completing synchronization of the activity monitor 342 therewith. In some embodiments, the centralized health application 306 can provide a notification 506 that synchronization has been completed, for example, via a popup or overlay message.

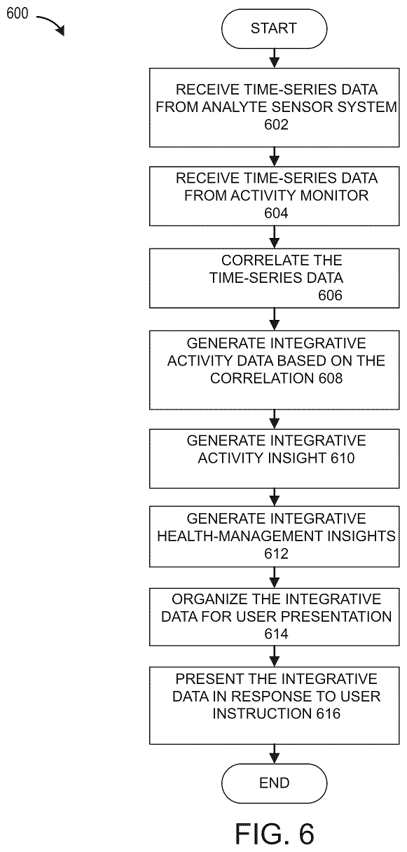

FIG. 6 illustrates an example of a process 600 for integrating health data from multiple wearables. In some embodiments, the process 600 can be executed, for example, by the central health engine 312 of FIG. 3 and/or the central health engine 112 of FIGS. 1A-B and 2. In addition, or alternatively, the process 600 can be executed, for example, by the application 106 of FIGS. 1A-B and 2 or the centralized health application 306 of FIG. 3. In addition, or alternatively, the process 600 can be executed generally by the display device 307 of FIG. 3, any of the display devices 107 of FIGS. 1A-B and 2, and/or any of the remote systems 325 of FIG. 3. Although any number of systems, in whole or in part, can implement the process 600, to simplify discussion, the process 600 will be described primarily in relation to the central health engine 312 of FIG. 3.

At block 602, the central health engine 312 receives time-series data from the analyte sensor system 304, where the time-series data can include any time-series data acquired or generated by the analyte sensor system 304. For example, the time-series data can include a time series of analyte levels of a user of the analyte sensor system 304, as described relative to FIG. 3. In certain embodiments, the time-series data can include multiple time series, as also described relative to FIG. 3. In some embodiments, some or all of the time-series data can be received on a continuous basis (e.g., every 30 seconds, every 5 minutes, every 10 minutes, etc.).

At block 604, the central health engine 312 receives time-series data from the activity monitor 342, where the time-series data can include any time-series data acquired or generated by the activity monitor 342, such as the activity information discussed relative to the metrics 130 of FIG. 2. For example, the time-series data can include activity information that identifies one or more recorded activities of one or more defined activity types, as described relative to FIG. 3. In certain embodiments, the time-series data from the activity monitor 342 can include multiple time series, as also described relative to FIG. 3. In some embodiments, some or all of the time-series data from the activity monitor 342 can be received from the activity monitor 342 on a continuous basis. In addition, or alternatively, the time-series data can be received from the activity monitor 342 in response to user-initiated synchronization of the activity monitor 342 with the centralized health application 306, for example, as described relative to FIGS. 4 and 5.

At block 606, the central health engine 312 correlates the time-series data from the analyte sensor system 304 and the time-series data from the activity monitor 342 based on time. In an example, if the time series from the analyte sensor system 304 includes analyte levels (e.g., glucose levels) of the user and the time series from the activity monitor 342 includes activity information that identifies a recorded activity of a defined activity type, the central health engine 312 can correlate the analyte levels to the activity based on a time period of the activity, as discussed previously. In another example, the central health engine 312 can correlate values of a time series from the analyte sensor system 304, such as glucose levels, with values of a time series from the activity monitor 342, such as heart rate or GPS data, using timestamps associated with the values. In some embodiments, the correlation can involve mapping different time series to a common representation of time, for example, so as to harmonize different sampling intervals.

In some embodiments, as discussed previously, the time-series data from the activity monitor 342 can identify recorded activities of one or more defined activity types (e.g., walk, run, sprint, swim, weightlift, etc.), with each activity being associated with a duration and/or time period (e.g., 30 minutes or 5:00-5:30 pm). In some of these embodiments, with respect to a given activity (e.g., a 3-mile run), the central health engine 312 can correlate values of one time series from the analyte sensor system 304, such as analyte concentrations, with values of another time series from the activity monitor 342, such as heart rate or GPS data, based on a span of time that includes the duration and/or time period of the activity (e.g., 30 minutes or 7:00-7:30 am). For example, the values of the two time series can be related to a common representation of time that includes the span of time. In some cases, the span of time can include a configurable period of time before and/or after the duration and/or time period of the activity (e.g., 6:30-8:00 am for an activity that spans 7:00-7:30 am), such that values before and/or after the activity are available for comparative presentation and analysis.

At block 608, the central health engine 312 generates integrative activity data, such as the integrative activity data discussed relative to the metrics 130 of FIG. 2, based on the correlation at the block 606. The integrative activity data can include any combination of data from the correlated time-series data. In addition, or alternatively, the integrative activity data can include any data that is generated based on a summarization, transformation, or analysis of any time-series data from the two wearables. Examples of integrative activity data will be shown and described relative to FIGS. 7A-B, 8A-C, 9, 10, and 12-19.

For example, if the block 606 results in the central health engine 312 correlating values of a time series from the analyte sensor system 304 with values of a time series from the activity monitor 342 based on time, the block 608 can involve generating a visualization based thereon, which is an example of integrative activity data. The visualization can be a graphical control element that includes, for example, a graph. In various cases, the graphical control element can plot the two parameters over time and/or compare values of the two parameters over time. An example of the visualization will be described relative to FIGS. 7A, 9, and 14-19.

As mentioned previously, in some embodiments, the correlation at the block 606 can be based on a span of time that includes a duration and/or time period of a defined activity, such as walking, running, swimming, weightlifting, or the like. Also as discussed previously, the span of time can include a configurable period of time before and/or after the defined activity. In these examples, the graphical control element, such as a graph, can plot the two parameters over the span of time and/or compare values of the two parameters over the span of time. In addition, or alternatively, the integrative activity data can include, for the duration and/or time period of the activity, a plot of analyte levels, a lowest analyte level recorded, a highest analyte level recorded, a time in range, average analyte level, and/or indications of any analyte events that occurred, as further discussed below. An example of integrative activity data for recorded activities will be described relative to FIGS. 7A-B, 8A-C, 9, 10, and 12-19.

As another example, in some embodiments, the block 608 can include the central health engine 312 using the time-series data from the analyte sensor system 304 to identify analyte events during an activity identified in the time-series data from the activity monitor 342. The analyte events can include, for example, an analyte concentration above an upper threshold at a given time during the activity (i.e., a high analyte level), an analyte concentration below a lower threshold at a given time during the activity (i.e., a low analyte level), a rate of change of analyte concentration in excess of a threshold (i.e., a rapidly changing analyte level), and/or the like. An example of such an analyte event will be described relative to FIG. 7B.

In some embodiments, the block 608 can further include correlating any identified analyte events to a time series of the time-series data provided by the activity monitor 342. For example, an analyte event, such as a high, low, or rapidly changing analyte level, can be correlated to GPS data, heart rate data, and/or other data included in the time-series data provided by the activity monitor 342 based on a time of the analyte event. In the case of GPS or heart rate data, the analyte event can be correlated based on time, such that the GPS data to which the event is correlated is indicative of a location or locations at which the analyte event occurred, and such that the heart rate data to which the event is correlated is indicative of heart rate at or around the time of the event. An example of integrative activity data that includes an analyte event correlated to time-series data from the activity monitor 342 will be described relative to FIG. 7B.

At block 610, the central health engine 312 generates integrative activity insights, such as the integrative activity insights discussed relative to the metrics 130 of FIG. 2. In various embodiments, the central health engine 312 can generate the integrative activity insights based on the integrative activity data from the block 608. The integrative activity insights can include, for example, recommendations or advice related to future activities or behaviors of the user, where the recommendations or advice is based on values and/or relationships between values in the integrative activity data.

In certain embodiments, the integrative activity insights generated at the block 610 can be based on analyte events of the type described relative to the block 608. For example, if the central health engine 312 has identified, at the block 608, an analyte event at a given time (e.g., a low analyte concentration at 3:01 pm during a run), the central health engine 312 can utilize the integrative activity data to identify, based on stored configurations, preconfigured possible triggers of the event (e.g., hill, increased speed, etc.), validate the existence of the triggers relative to the event (e.g., determine that the event occurred while the user was climbing a hill potentially in combination with other criteria such as elevated heart rate), and generate a recommendation related to the trigger (e.g., preconfigured advice such as “slow down” or “lower heart rate” at or around the location where the event occurred). Examples of integrative activity insights will be described relative to FIGS. 7C-D.

At block 612, the central health engine 312 generates integrative health-management insights, such as the integrative health-management insights discussed relative to the metrics 130 of FIG. 2. For an overall health dataset, such as for any combination of the inputs 127 and/or others of the metrics 130 of FIG. 2, the central health engine 312 can generate integrative health-management insights, such as diabetes-management insights. The overall health dataset can include, for example, the time-series data received from the analyte sensor system at the block 602, the time-series data received from the activity monitor 342 at the block 604, the integrative activity data from the block 608, the integrative activity insights from the block 610, and/or other data. In some embodiments, the overall health dataset can include, for example, the foregoing types of data resultant from multiple iterations of the process 600.

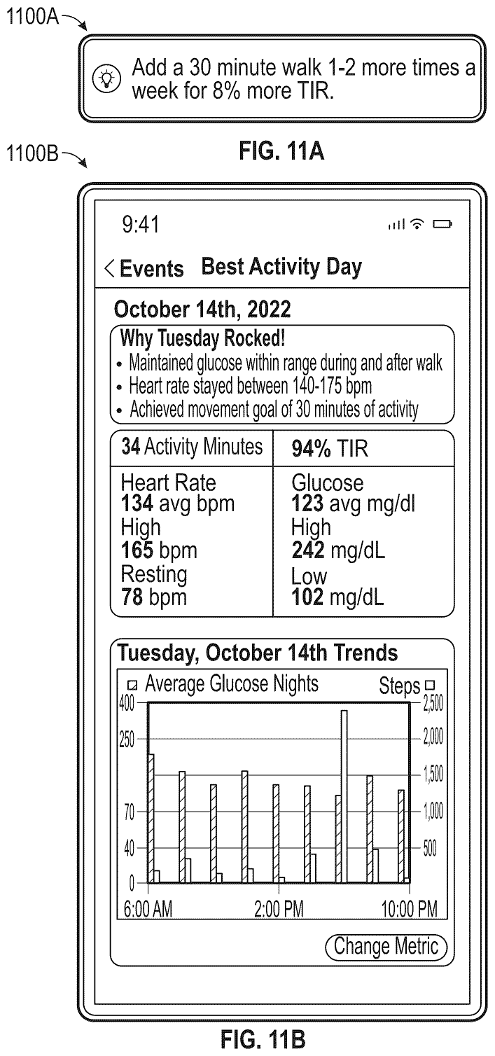

Still with reference to the block 612, in some embodiments, the integrative health-management insights can include activity recommendations for the user based on, for example, the overall health dataset above. For example, the central health engine 312 can analyze the integrative activity data over a configurable period of time (e.g., 14 days), identify, based on stored configurations, an activity that represents an incremental addition to the user's current activity level (e.g., add a 30-minute walk 1-2 more times per week), and determine, based on the stored configurations, a statistical benefit of adding the activity to the user's current activity level (e.g., 8% more glucose time in range). According to this example, the identified activity and the determined statistical benefit may be indicated or provided as a health-management insight. An example of such an integrative health-management insight will be described relative to FIG. 11A.

Still referring to the block 612, in some embodiments, the integrative health-management insights can include identification of noteworthy days, weeks, or other periods of time. For example, the central health engine 312 can analyze integrative activity data for a period of time, such as a week, and evaluate such data. According to this example, the central health engine 312 can compare activity and/or analyte data for each day over the period of time to criteria specified in stored configurations to evaluate the day (e.g., deem the day good, bad, acceptable, etc.), identify a noteworthy (e.g., best or worst) day over the period, and/or the like. In a more particular example, the central health engine 312 can identify the best day based on criteria that defines or rates how days may be deemed superior to other days. The criteria can include, for example, whether or a degree to which analyte levels were in range during the day's activities (e.g., during the activities and/or for a specified period after the activities), whether or a degree to which heart rate was in range during the activities, and whether or a degree to which the day's movement goal was achieved (e.g., 30 minutes of activity). An example of such an integrative health-management insight will be described relative to FIG. 11B.

At block 614, the central health engine 312 organizes integrative data for user presentation. The integrative data can include, for example, any of the data generated at the blocks 608, 610, and/or 612. The integrative data can be organized into preconfigured and/or configurable user interface containers of related data, such as cards. For example, for individual activities or groups of activities, the central health engine 312 can organize integrative activity data and/or integrative activity insights into user interface containers of related data, such as cards, for presentation to the user. The user interface containers can allow for customization of the included data.

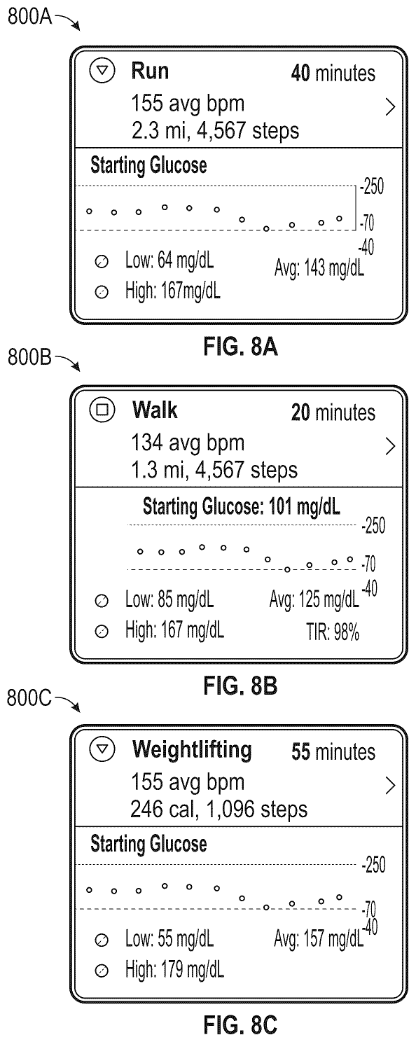

In some embodiments, the block 614 can include the central health engine 312 generating a user interface container, such as a glanceable activity card, that includes integrative activity data for an activity. The integrative activity data can include, for example, a glucose plot over the activity, lowest glucose value recorded, highest glucose value recorded, time in range, average glucose, indications of any analyte events that occurred, and/or other data. Examples of glanceable activity cards will be described relative to FIGS. 8A-C.

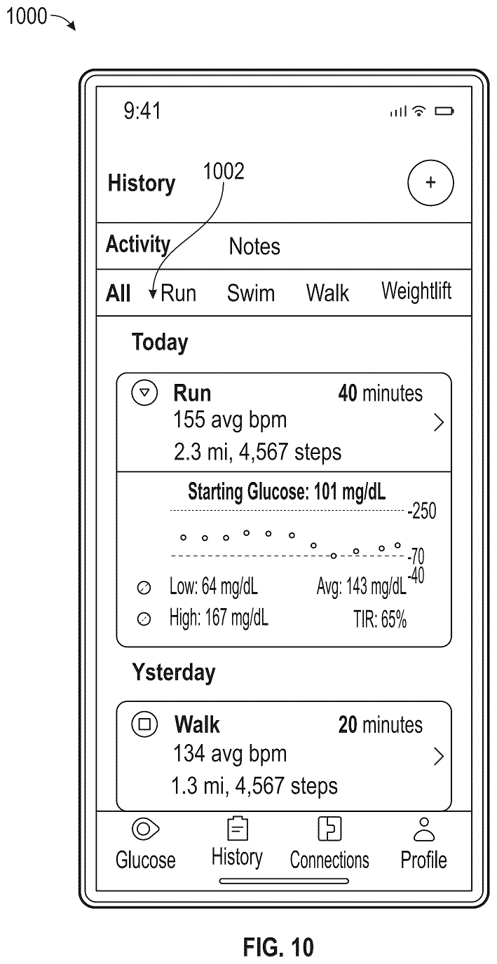

In some embodiments, the organization at the block 614 can be triggered by a user request for particular data. For example, for any desired data view, such as overall health, groups of activities, selected time periods, and/or the like, as part of the block 614, the central health engine 312 can aggregate multiple sets of integrative activity data and/or integrative activity insights for the requested or desired view. In response to such a request, the central health engine 312 can organize the aggregated data into user interface containers of related data, such as cards, for presentation to the user. An example of organizing data into user interface containers based on a desired view will be described relative to FIG. 10.

At block 616, the central health engine 312 presents the organized integrative data to the user, for example, in response to user request or instruction, or as otherwise desired. Examples of user interface views that can be presented at the block 616 will be described relative to FIGS. 7A-D, 8A-C, and 9-19. After block 616, the process 600 ends.

For illustrative purposes, the process 600 is described as having a sequential flow. It should be appreciated, however, that the blocks thereof can be performed in any order and/or selectively omitted. Certain blocks of the process 600 can also be performed less frequently and/or independently. For example, the generation of integrative activity insights at the block 610 may be performed less frequently than the generation of integrative activity data, for example. In a similar fashion, the generation of integrative health-management data at the block 612 may be performed less frequently than the other blocks. In some cases, the block 610 and/or the block 612 may be performed independently relative to the rest of the process 600. Other examples and variations will be apparent to one skilled in the art after a detailed review of the present disclosure.

For illustrative purposes, examples of certain aspects of the process 600 will now be described relative to embodiments in which the analyte sensor system 304 is a CGM that estimates a blood glucose concentration level from a measurement of subcutaneous fluid. Therefore, various examples may be described in terms of estimated blood glucose concentration levels and glucose events that are defined relative to those levels. It should be appreciated, however, that the methods and systems described herein may also be applied to other types of concentration levels and other types of events that are defined in relation to those other types of concentration levels. For example, the other types of concentration levels can be actual blood glucose concentration levels or levels of other analytes in blood or other body fluids or substances. The methods and systems described herein may also be applied to other physiologic states or conditions for which management is desirable for health or other reasons.

FIG. 7A illustrates an example of a user interface view 700A that presents integrative activity data for a recorded activity. The integrative activity data may be generated, organized, and presented as described, for example, relative to the blocks 608, 614, and 616, respectively, of FIG. 6. In the example of FIG. 7A, the recorded activity is a 45-minute run.

In the illustrated embodiment, the user interface view 700A includes a graphical control element in the form of a graph that compares glucose levels 702A from the analyte sensor system 304 with heart rates 704A from the activity monitor 342 and an event stream 705A. The glucose levels 702A and the heart rates 704A are plotted over a span of time that includes both time before and time after the recorded activity. The event stream 705A is shown as a swim lane that includes various events such as the 45-minute run, meals, calibrations, or other events (e.g., user-inputted events as shown in FIG. 5).

In various embodiments, the time series that are compared in the user interface view 700A can be dynamically customized by a user, for example, via a “change health metric” option 706A, which is shown as a button in the example of FIG. 7A. For example, the user can press or activate the “change health metric” option 706A and then select or configure two or more time series for comparison, for example, from the inputs 127 and/or the metrics 130 of FIG. 2. The user selection and/or configuration of the two or more time series can serve as a user instruction to change the user interface view 700A, for example, by replacing the current comparison with a comparison of the two or more time series. In some cases, the two or more time series that are selected or configured via the “change health metric” option 706A may be entirely different from the time series already shown in user interface view 700A. In other cases, at least one of the two or more time series may be the same as a time series already shown in the user interface view 700A. The central health engine 312 can receive the user instruction and, in response, automatically generate a new graphical control element that plots the selected or configured two or more time series over the span of time. The central health engine 312 can automatically update the user interface view 700A with the new graphical control element, or otherwise automatically cause the new graphical control element to be displayed.

FIG. 7B illustrates an example of a user interface view 700B that presents additional integrative activity data for the recorded activity discussed relative to FIG. 7A. The additional integrative activity data may be generated, organized, and presented as described, for example, relative to the blocks 608, 614, and 616, respectively, of FIG. 6. In the example of FIG. 7B, the user interface view 700B is an activity card that reports an identified glucose event (i.e., low glucose) which has been correlated to a location where the glucose event occurred (i.e., “Main Street hill”).

FIG. 7C illustrates an example of a user interface view 700C that presents an integrative activity insight for the recorded activity discussed relative to FIG. 7A. The integrative activity insight may be generated, organized, and presented as described, for example, relative to the blocks 610, 614, and 616, respectively, of FIG. 6. For illustrative purposes, the integrative activity insight shown in the user interface view 700C is based on the identified analyte event reported in the user interface view 700B of FIG. 7B. In the example of FIG. 7C, the user interface view 700C is an activity card that recommends walking or slowing heart rate at the location where the identified analyte event from FIG. 7B occurred (i.e., “Main Street hill”).

FIG. 7D illustrates an example of a user interface view 700D that presents an additional integrative activity insight for the recorded activity discussed relative to FIG. 7A. The additional integrative activity insight may be generated, organized, and presented as described, for example, relative to the blocks 610, 614, and 616, respectively, of FIG. 6. For illustrative purposes, the integrative activity insight shown in the user interface view 700D is based on an identified glucose event during the recorded activity, specifically, a low glucose event. In the example of FIG. 7D, the user interface view 700D is an activity card that, as the integrative activity insight, recommends reducing heart rate to prevent another similar glucose event from occurring in the future.

FIGS. 8A-C illustrate examples of user interface views that present integrative activity data for different types of recorded activity. The integrative activity data may be generated, organized, and presented as described, for example, relative to the blocks 608, 614, and 616, respectively, of FIG. 6.

FIG. 8A illustrates an example of a user interface view 800A that is a glanceable activity card for a run. In the example of FIG. 8A, the integrative activity data presented by the user interface view 800A includes, for a duration of the run, an average heart rate, a distance, a step count, a plot of glucose levels, a lowest glucose level recorded, a highest glucose level recorded, and an average glucose level.

FIG. 8B illustrates an example of a user interface view 800B that is a glanceable activity card for a walk. In the example of FIG. 8B, the integrative activity data presented by the user interface view 800B includes, for a duration of the walk, an average heart rate, a distance, a step count, a plot of glucose levels, a lowest glucose level recorded, a highest glucose level recorded, an average glucose level, and a glucose time in range.

FIG. 8C illustrates an example of a user interface view 800C that is a glanceable activity card for weightlifting. In the example of FIG. 8C, the integrative activity data presented by the user interface view 800C includes, for a duration of the weightlifting, an average heart rate, a distance, a step count, a plot of glucose levels, a lowest glucose level recorded, a highest glucose level recorded, and an average glucose level.

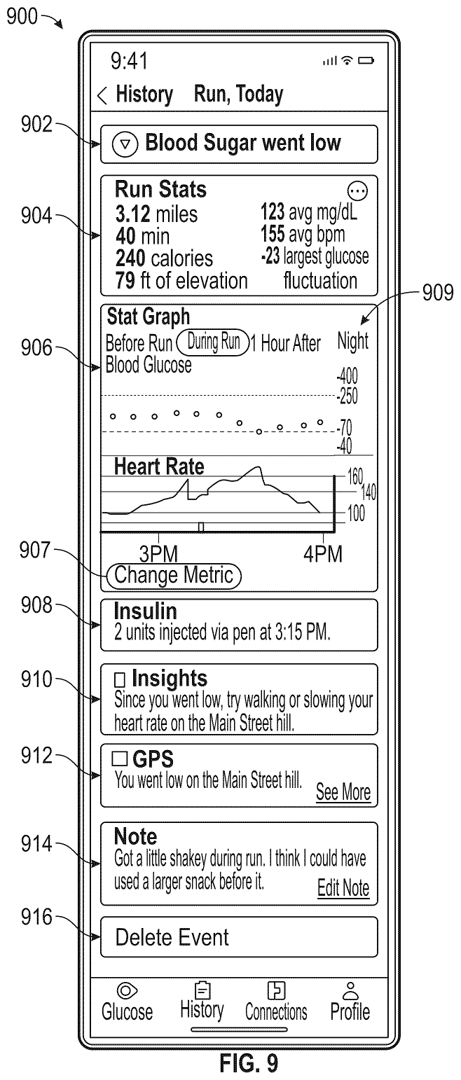

FIG. 9 illustrates an example of a user interface view 900 that presents integrated data for a recorded activity. In the example of FIG. 9, the recorded activity is a run. The user interface view 900 includes a configurable selection of user interface containers, shown as cards, that each present collections of related data for the run. In some embodiments, the user interface view 900 may serve as a detailed workout summary that integrates time-series data from both the analyte sensor system 304 and the activity monitor 342.

Still referring to FIG. 9, the user interface view 900 includes an example selection of cards that present integrative activity data related to the run, namely, a glucose event card 902, an activity statistics card 904, a statistics graph card 906, an insulin card 908, and a GPS integration card 912. The integrative activity data shown in the user interface view 900 can be generated, for example, as part of the block 608 of FIG. 6 and organized into cards, for example, as part of the block 614 of FIG. 6.

The glucose event card 902 presents an identified glucose event during the run (i.e., a low glucose event). The activity statistics card 904 presents a selection of activity statistics based on information from the analyte sensor system 304 (e.g., average glucose level and largest glucose fluctuation) and/or the activity monitor 342 (e.g., miles, minutes, calories, elevation, and average heart rate). The statistics graph card 906 shows plots of glucose levels and heart rate over a span of time that includes the run. The insulin card 908 can present information related to insulin doses delivered before, during, and/or after the activity. The GPS integration card 912 presents results of a time-based correlation between glucose values and GPS data as described relative to FIG. 6. The GPS integration card 912 presents results of a time-based correlation between glucose values and GPS data as described relative to the block 608 of FIG. 6 and FIG. 7B.

Still with reference to the example of FIG. 9, the user interface view 900 also includes a card that presents an integrative activity insight related to the run, namely, an insight card 910. The integrative activity insight can be generated, for example, as part of the block 610 of FIG. 6. The example integrative activity insight presented in the insight card 910 is based on the time-based correlation between glucose values and GPS data, as described relative to the block 610 of FIG. 6 and FIG. 7C.

Still with reference to the example of FIG. 9, the user interface view 900 further includes a notes card 914 and a management options card 916. The notes card 914 presents user notes on the run and provides an option for user editing. The management options card 916 presents management options such as an option to delete the run.

In some embodiments, the user interface view 900 can be dynamically modified or updated in response to user instruction. For example, in various embodiments, the time series that are compared in the statistics graph card 906 can be dynamically customized by a user, for example, via a “change metric” option 907, which is shown as a button in the example of FIG. 9. For example, the user can press or activate the “change metric” option 907 and then select or configure two or more time series for comparison, for example, from the inputs 127 and/or the metrics 130 of FIG. 2. The user selection and/or configuration of the two or more time series can serve as a user instruction to change the statistics graph card 906, for example, by replacing the current comparison with a comparison of the two or more time series. In some cases, the two or more time series that are selected or configured via the “change metric” option 907 may be entirely different from the time series already shown in the statistics graph card 906. In other cases, at least one of the two or more time series may be the same as a time series already shown in the statistics graph card 906. The central health engine 312 can receive the user instruction and, in response, automatically generate a new graphical control element that plots the selected or configured two or more time series over the span of time. The central health engine 312 can automatically update the statistics graph card 906 with the new graphical control element, or otherwise automatically cause the new graphical control element to be displayed.

By way of further example, still with reference to the statistics graph card 906, the span of time over which the time series are compared can be dynamically customized or changed by the user, for example, via time change option 909. In the example of FIG. 9, the time change option 909 includes a plurality of time choices, namely, a period before the run (e.g., four hours before the run begins), the time period of the run, a period after the run (e.g., two hours after the run), and overnight after the run (e.g., 11:00 pm-7:00 am following the run). In some embodiments, the user can directly specify the span of time. Other examples for the time choices will be apparent to one skilled in the art after a detailed review of the disclosure. According to this example, the time choice selected by the user can serve as a user instruction to change the span of time represented in the statistics graph card 906. The central health engine 312 can automatically generate a new graphical control element that plots the time series over the changed span of time. The central health engine 312 can automatically update the statistics graph card 906 with the new graphical control element, or otherwise automatically cause the new graphical control element to be displayed.

FIG. 10 illustrates an example of a user interface view 1000 that can include activity cards for selectable groups of activities, such as historical activities. In general, for groups of activities, such as activities occurring over a selected period of time, the central health engine 312 can generate and provide a scrollable user interface view, similar to the user interface view 1000, that includes a user interface container for each activity over the selected period of time. For each activity, the user interface container can be glanceable activity card that includes, for example, integrative activity data for the activity. The integrative activity data may be generated, organized, and presented as described, for example, relative to the blocks 608, 614, and 616, respectively, of FIG. 6.

Still referring to FIG. 10, the user interface view 1000 is scrollable and includes filter options 1002 for filtering by type of activity. In the example of FIG. 10, the filter options 1002 are user-selectable and include options for selecting all activities, only running activities, only swimming activities, only walking activities, or only weightlifting activities. Further, in the example of FIG. 10, the filter options 1002 indicate that all activities are selected for inclusion in the user interface view 1000.

For illustrative purposes, the example groups of activities and corresponding filter options shown in FIG. 10 are related to sports-like exercise. However, it should be appreciated that, in various embodiments, the groups and filter options may include activities that are not specifically related to sports-like exercise. For example, in some embodiments, the groups of activities and filter options can include gardening, mowing, playing a musical instrument, or the like. In another example, in some embodiments, such non-sports and/or non-exercise activities can be combined into a single group that is selectable as a filter option.

FIG. 11A illustrates an example of a user interface view 1100A that presents an integrative health-management insight. The integrative health-management insight may be generated, organized, and presented as described, for example, relative to the blocks 612, 614, and 616, respectively, of FIG. 6.

FIG. 11B illustrates an example of a user interface view 1100B that presents an integrative health-management insight. The integrative health-management insight may be generated, organized, and presented as described, for example, relative to the blocks 612, 614, and 616, respectively, of FIG. 6. In particular, in the example of FIG. 11B, a best activity day over a period such as a week is identified to the user.

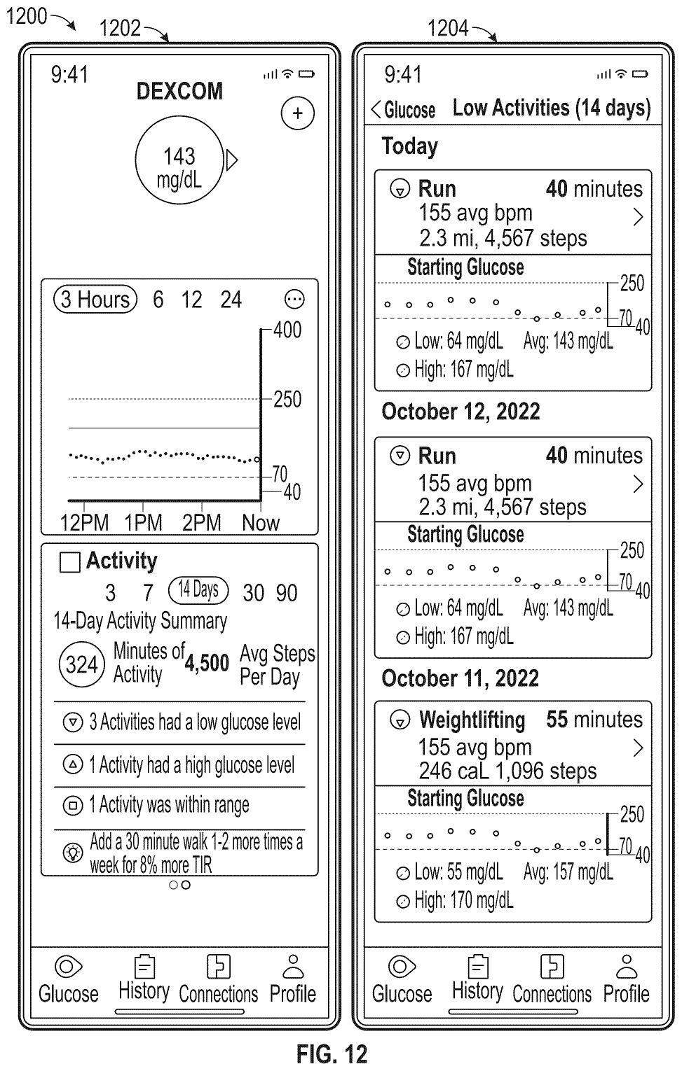

FIG. 12 illustrates an example of user interface views 1200 that present integrative activity data for a configurable period, such as a preceding 14 days. The integrative activity data may be generated, organized, and presented as described, for example, relative to the blocks 608, 614, and 616, respectively, of FIG. 6. The user interface views 1200 include a first view 1202 and a second view 1204.

In the example of FIG. 12, the first view 1202 indicates, for a selected 14-day period, how many activities had a low-glucose event, how many had a high-glucose event, and how many had no glucose events such that glucose was in range. The first view 1202 allows the user to drill down into activities according to glucose-level classification.

According to the example of FIG. 12, the user selects, from the first view 1202, the activities with the low glucose level, which results in the centralized health application presenting the second view 1204. The second view 1204 illustrates an example of a scrollable user interface view that can include activity cards for the activities with the low glucose level.

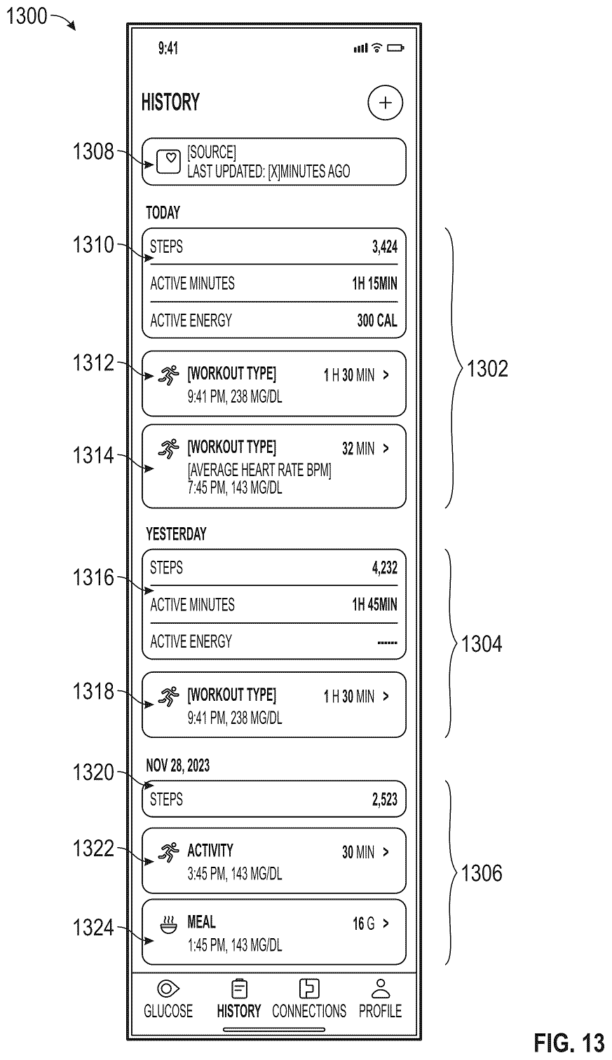

FIG. 13 illustrates an example of a user interface view 1300 that presents integrative activity data for a configurable period, such as a period covering a current day and the two preceding days. The integrative activity data may be generated, organized, and presented as described, for example, relative to the blocks 608, 614, and 616, respectively, of FIG. 6.

In the example of FIG. 13, the user interface view 1300 includes a day section 1302 corresponding to a current day (i.e., “today”), a day section 1304 corresponding to the previous day (i.e., “yesterday”), and a day section 1306 corresponding to the day before the previous day (i.e., the day before “yesterday”). The day sections 1302, 1304, and 1306 can each include a configurable selection of user interface containers, shown as cards, that each present collections of data for the corresponding day. The user interface view 1300 further includes synchronization card 1308 indicating, for example, when the display device 307 most recently synchronized with the activity monitor 342.

Continuing the example of FIG. 13, the day section 1302 includes a daily summary card 1310, an activity statistics card 1312, and an activity statistics card 1314. The daily summary card 1310 summarizes activity during the current day (e.g., step count, number of active minutes, etc.). The activity statistics card 1312 and the activity statistics card 1314 each present a selection of activity statistics based on information from the analyte sensor system 304 (e.g., average glucose level) and/or the activity monitor 342 (e.g., minutes and average heart rate).

Similarly, the day section 1304 includes a daily summary card 1316 and an activity statistics card 1318. The daily summary card 1316 summarizes activity during the previous day (e.g., step count, number of active minutes, etc.). The activity statistics card 1318 presents a selection of activity statistics based on information from the analyte sensor system 304 (e.g., average glucose level) and/or the activity monitor 342 (e.g., minutes and average heart rate).

The day section 1306 includes a daily summary card 1320, an activity statistics card 1322, and a meal card 1324. The daily summary card 1320 summarizes activity two days prior to the current day (e.g., step count). As illustrated, in some embodiments, the daily summary card 1320 may be less detailed or otherwise different from the daily summary cards 1310 and 1316 for the current day and the previous day, respectively. The activity statistics card 1322 presents a selection of activity statistics based on information from the analyte sensor system 304 (e.g., average glucose level) and/or the activity monitor 342 (e.g., minutes and average heart rate). The meal card 1324 presents data related to a meal (e.g., amount of carbs and a glucose level at the time of the meal).

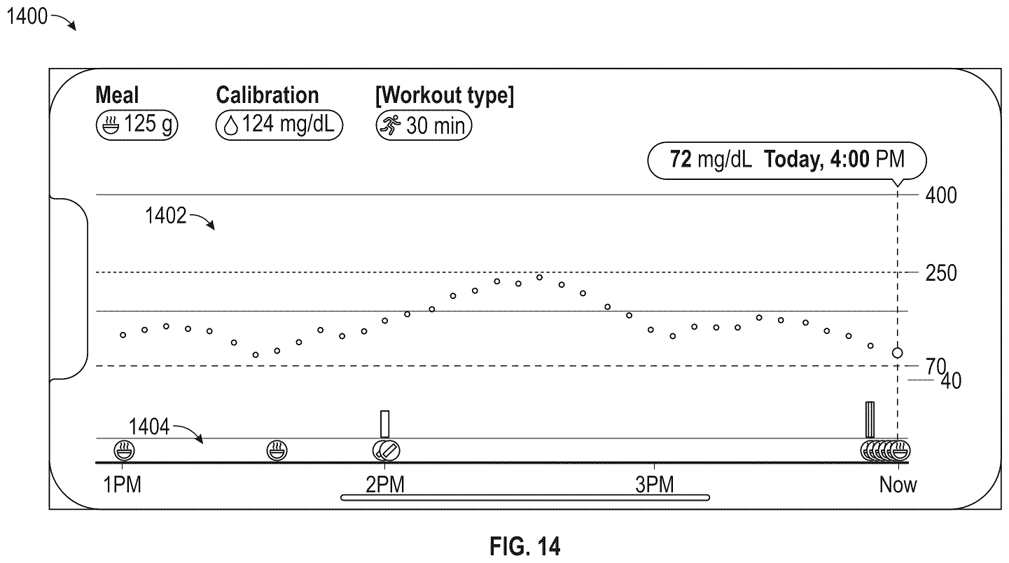

FIG. 14 illustrates an example of a user interface view 1400 that presents integrative activity data for a recorded activity. The integrative activity data may be generated, organized, and presented as described, for example, relative to the blocks 608, 614, and 616, respectively, of FIG. 6. In the example of FIG. 14, the recorded activity is a 30-minute run.

In the illustrated embodiment, the user interface view 1400 includes a graphical control element in the form of a graph that compares glucose levels 1402 from the analyte sensor system 304 with an event stream 1404. The glucose levels 1402 and the event stream 1404 are plotted over a span of time that includes both time before and time after the recorded activity. The event stream 1404 is shown as a swim lane that includes various events such as the 30-minute run, meals, calibrations, or other events (e.g., user-inputted events as shown in FIG. 5).

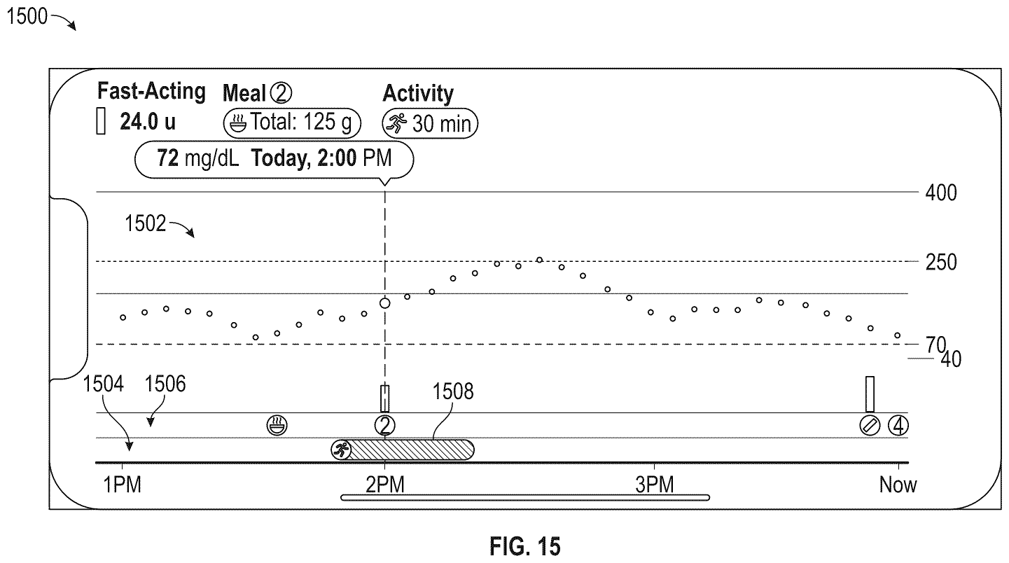

FIG. 15 illustrates an example of a user interface view 1500 that presents integrative activity data with multiple event streams. The integrative activity data may be generated, organized, and presented as described, for example, relative to the blocks 608, 614, and 616, respectively, of FIG. 6. The user interface view 1500 includes a graphical control element in the form of a graph that compares glucose levels 1502 from the analyte sensor system 304 with an event stream 1504 and an event stream 1506.

In the example of FIG. 15, the recorded activity is a 30-minute run, and the glucose levels 1502, the event stream 1504, and the event stream 1506 are plotted over a span of time of approximately 1:00-4:00 pm. In the example of FIG. 15, the span of time includes both time before and time after the recorded activity. The event stream 1504 is shown as a swim lane that illustrates activities during the span of time, including a 30-minute duration 1508 of the recorded 30-minute run. The event stream 1506 is shown as a separate swim lane that illustrates non-activity events over the span of time. The non-activity events can include, for example, meals, calibrations, and/or other events (e.g., user-inputted events as shown in FIG. 5).

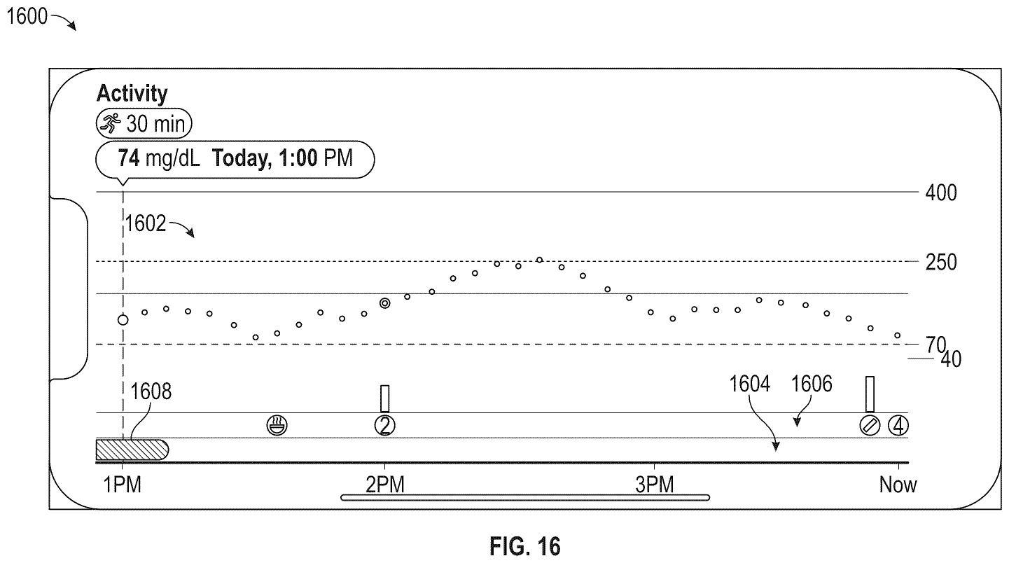

FIG. 16 illustrates another example of a user interface view 1600 that presents integrative activity data with multiple event streams. The integrative activity data may be generated, organized, and presented as described, for example, relative to the blocks 608, 614, and 616, respectively, of FIG. 6. The user interface view 1600 includes a graphical control element in the form of a graph that compares glucose levels 1602 from the analyte sensor system 304 with an event stream 1604 and an event stream 1606.

Similar to FIG. 15, in the example of FIG. 16, the recorded activity is a 30-minute run, and the glucose levels 1602, the event stream 1604, and the event stream 1606 are plotted over a span of time of approximately 1:00-4:00 pm. Different from FIG. 15, in the example of FIG. 16, the plotted span of time begins during the recorded 30-minute activity. The event stream 1604 is shown as a swim lane that illustrates activities such as, for example, a partial duration 1608 of the recorded 30-minute run. The event stream 1606 is shown as a separate swim lane that illustrates non-activity events over the span of time.

FIG. 17 illustrates an example of a user interface view 1700 that presents integrative activity data for overlapping activities. The integrative activity data may be generated, organized, and presented as described, for example, relative to the blocks 608, 614, and 616, respectively, of FIG. 6. The user interface view 1700 includes a graphical control element in the form of a graph that compares glucose levels 1702 from the analyte sensor system 304 with an event stream 1704 and an event stream 1706.

In the example of FIG. 17, two recorded activities are shown, namely, a 30-minute activity and a 10-minute activity, such that the two recorded activities overlap. In particular, the two recorded activities have the same start time but different end times. In some embodiments, the two recorded activities, and the corresponding overlap, may result from a difference in activity detection between two or more devices. For example, the activity monitor 342 may detect or identify a 10-minute walk or run, while the display device 307 may detect or identify a 30-minute walk or run. In addition, or alternatively, the two recorded activities may be related such that a more specific activity occurs within a broader activity. For example, a 30-minute run may include a 10-minute sprint. In additionally, or alternatively, the two activities can be separate activities that occur at the same time, such as strength training concurrent with cardiovascular exercise. Other examples will be apparent to one skilled in the art after a detailed review of the present disclosure.

Similar to FIGS. 15 and 16, in the example of FIG. 17, the glucose levels 1702, the event stream 1704, and the event stream 1706 are plotted over a span of time of approximately 1:00-4:00 pm. The event stream 1704 is shown as a swim lane that illustrates activities, including a duration 1708 of the recorded 30-minute activity and a duration 1710 of the recorded 10-minute activity. The event stream 1706 is shown as a separate swim lane that illustrates non-activity events over the span of time.

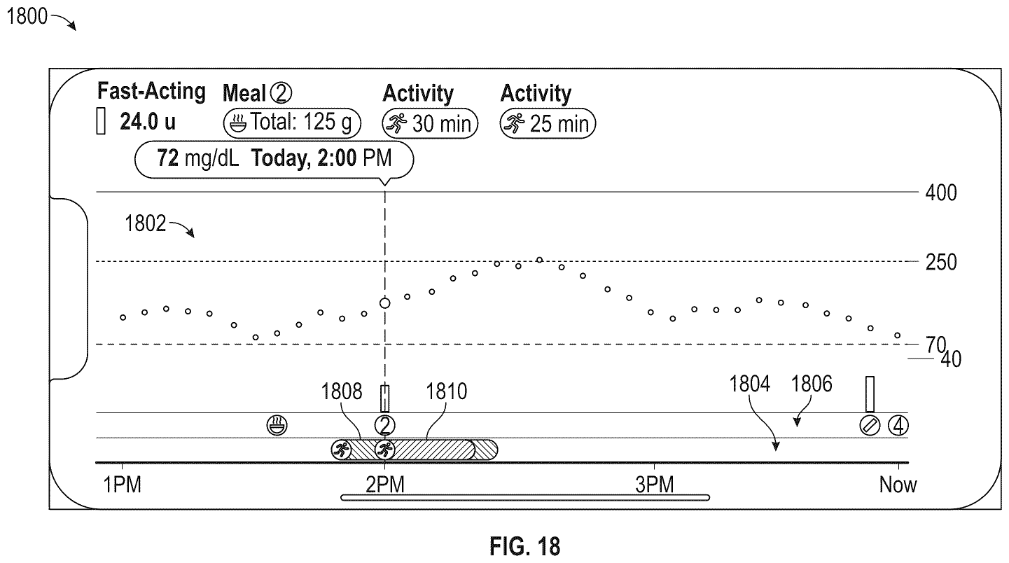

FIG. 18 illustrates another example of a user interface view 1800 that presents integrative activity data for overlapping activities. The integrative activity data may be generated, organized, and presented as described, for example, relative to the blocks 608, 614, and 616, respectively, of FIG. 6. The user interface view 1800 includes a graphical control element in the form of a graph that compares glucose levels 1802 from the analyte sensor system 304 with an event stream 1804 and an event stream 1806.

In the example of FIG. 18, two recorded activities are shown, namely, a 30-minute activity and a 25-minute activity, such that the two recorded activities overlap. In particular, the 25-minute activity occurs during the 30-minute activity, but the two recorded activities have different start and end times. In various embodiments, the two recorded activities can overlap, for example, for any of the reasons discussed relative to FIG. 17.

Similar to FIGS. 15-17, the glucose levels 1802, the event stream 1804, and the event stream 1806 are plotted over a span of time of approximately 1:00-4:00 pm. The event stream 1804 is shown as a swim lane that illustrates activities, including a duration 1808 of the recorded 30-minute activity and a duration 1810 of the recorded 25-minute activity. The event stream 1806 is shown as a separate swim lane that illustrates non-activity events over the span of time.

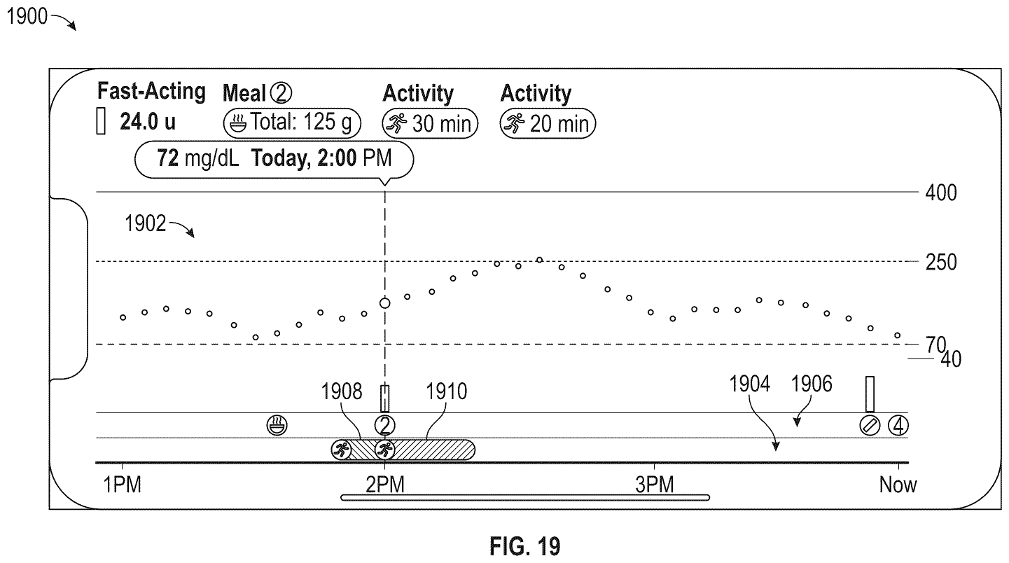

FIG. 19 illustrates another example of a user interface view 1900 that presents integrative activity data for overlapping activities. The integrative activity data may be generated, organized, and presented as described, for example, relative to the blocks 608, 614, and 616, respectively, of FIG. 6. The user interface view 1900 includes a graphical control element in the form of a graph that compares glucose levels 1902 from the analyte sensor system 304 with an event stream 1904 and an event stream 1906.

In the example of FIG. 19, two recorded activities are shown, namely, a 30-minute activity and a 20-minute activity, such that the two recorded activities overlap. In particular, the two recorded activities have different start times but the same end time. In various embodiments, the two recorded activities can overlap, for example, for any of the reasons discussed relative to FIG. 17.

Similar to FIGS. 15-18, in the example of FIG. 19, the glucose levels 1902, the event stream 1904, and the event stream 1906 are plotted over a span of time of approximately 1:00-4:00 pm. The event stream 1904 is shown as a swim lane that illustrates activities, including a duration 1908 of the recorded 30-minute activity and a duration 1910 of the recorded 20-minute activity. The event stream 1906 is shown as a separate swim lane that illustrates non-activity events over the span of time.

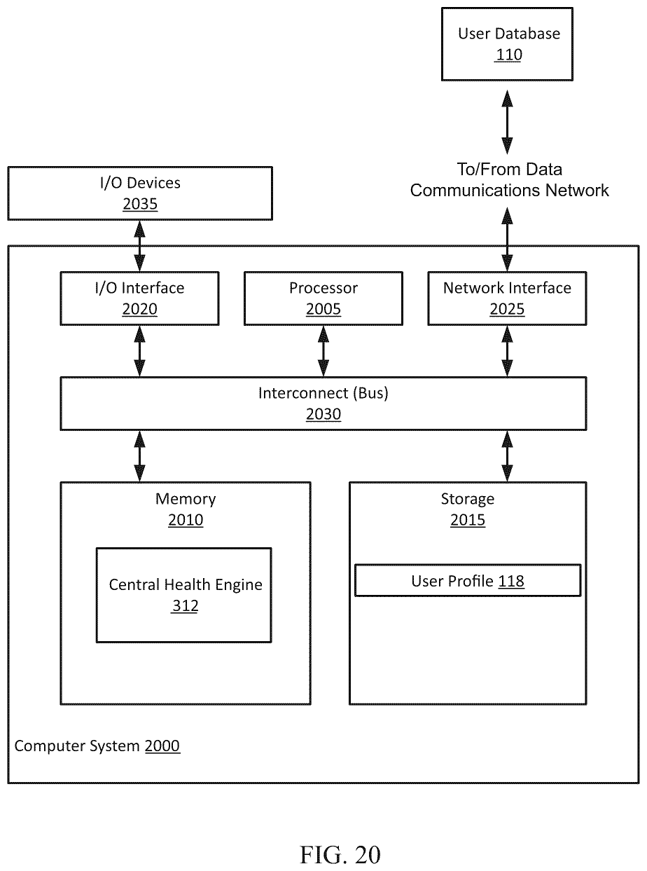

FIG. 20 is a block diagram depicting a computer system 2000 configured for integrating health data from multiple wearables, according to certain embodiments disclosed herein. Although depicted as a single physical device, in embodiments, the computer system 2000 may be implemented using virtual device(s), and/or across a number of devices, such as in a cloud environment. As illustrated, the computer system 2000 includes a processor 2005, a memory 2010, a storage 2015, a network interface 2025, and one or more I/O interfaces 2020. In the illustrated embodiment, the processor 2005 retrieves and executes programming instructions stored in the memory 2010, as well as stores and retrieves application data residing in the storage 2015. The processor 2005 is generally representative of a single CPU and/or GPU, multiple CPUs and/or GPUs, a single CPU and/or GPU having multiple processing cores, and the like.

The memory 2010 is generally included to be representative of a random access memory (RAM). The storage 2015 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, the I/O devices 2035 (such as keyboards, monitors, etc.) can be connected via the I/O interface(s) 2020. Further, via the network interface 2025, the computer system 2000 can be communicatively coupled with one or more other devices and components, such as the user database 110. In certain embodiments, the computer system 2000 is communicatively coupled with other devices via a network, which may include the Internet, local network(s), and the like. The network may include wired connections, wireless connections, or a combination of wired and wireless connections. As illustrated, the processor 2005, memory 2010, storage 2015, network interface(s) 2025, and the I/O interface(s) 2020 are communicatively coupled by one or more interconnects 2030. In certain embodiments, the computer system 2000 is representative of the display device 107 associated with the user. In certain embodiments, as discussed above, the display device 107 can include the user's laptop, computer, smartphone, and the like. In another embodiment, the computer system 2000 is a server executing in a cloud environment.

In the illustrated embodiment, the storage 2015 includes the user profile 118. The memory 2010 includes the central health engine 312. The central health engine 312 can executed by the computer system 2000 to perform operations, for example, of the process 600 for integrating health data from multiple wearable devices.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system for integrating health data from multiple wearables, the system comprising:
an analyte sensor system worn by a user;
an activity monitor worn by the user; and
a computer system configured to communicate with the analyte sensor system and the activity monitor, wherein the computer system is operable to:
receive, from the analyte sensor system worn by the user, a first time series of analyte levels of the user;
receive, from the activity monitor worn by the user, activity information that identifies an activity of a defined activity type;
correlate the analyte levels to the activity based on a time period of the activity; and
generate integrative activity data based on the correlation of the analyte levels to the activity.

2. The system of claim 1, wherein:
the first time series is received from the analyte sensor system on a continuous basis; and
the activity information is received in response to user-initiated synchronization of the activity monitor.

3. The system of claim 1, wherein the correlation is based on a span of time that includes at least one of a period of time before the time period of the activity or a period of time after the time period of the activity.

4. The system of claim 1, wherein the computer system is further operable to:
receive a second time series from the activity monitor worn by the user; and
correlate the first time series from the analyte sensor system and the second time series from the activity monitor based on the time period of the activity, wherein the generated integrative activity data is based on the correlated first time series and second time series.

5. The system of claim 4, wherein the second time series comprises at least one of heart rates, steps, or global positioning system (GPS) data.

6. The system of claim 4, wherein the generation of integrative activity data comprises generation of a graphical control element that compares the first time series with the second time series over a span of time comprising at least a portion of the time period of the activity.

7. The system of claim 6, wherein the computer system is further operable to cause the graphical control element to be displayed.

8. The system of claim 7, wherein the graphical control element comprises a change option, the computer system further operable to:
receive a user instruction, via the change option, to compare a third time series and a fourth time series over the span of time;
automatically generate a new graphical control element that compares the third time series with the fourth time series over the span of time; and
automatically cause the new graphical control element to be displayed.

9. The system of claim 8, wherein the third time series and the fourth time series are different from each of the first time series and the second time series.

10. The system of claim 7, wherein the graphical control element comprises an option to change a span of time over which the first time series and the second time series are compared, the computer system further operable to:
receive a user instruction, via the change option, to change the span of time;
automatically generate a new graphical control element that compares the first time series with the second time series over a changed span of time; and
automatically cause the new graphical control element to be displayed.

11. The system of claim 10, wherein the changed span of time comprises at least one of time before the time period of the activity or time after the time period of the activity.

12. The system of claim 1, wherein the generation of integrative activity data comprises identifying an analyte event during the time period of the activity based on the first time series.

13. The system of claim 12, wherein the analyte event relates to at least one of the analyte levels being below a threshold.

14. The system of claim 12, wherein:
the computer system is further operable to receive a second time series from the activity monitor worn by the user; and
the generation of integrative activity data further comprises correlating the analyte event to at least one parameter from the second time series based on a time of the analyte event.

15. The system of claim 14, wherein the at least one parameter comprises at least one of heart rate or GPS data.

16. The system of claim 12, wherein the computer system is further operable to generate an integrative activity insight based on the integrative activity data, wherein the integrative activity data comprises a recommendation for future activities of the user.

17. The system of claim 12, wherein the generation of an integrative activity insight comprises:
identifying a possible trigger of the analyte event based on stored configurations;
validating existence of the possible trigger relative to the analyte event; and
generating a recommendation related to the possible trigger responsive to the validating.

18. The system of claim 1, wherein the computer system is further operable to generate an integrative health-management insight based on the integrative activity data, the integrative health-management insight comprising an activity recommendation for the user.

19. The system of claim 18, wherein the generation of an integrative health-management insight comprises:
analyzing the integrative activity data over a period of time;
identifying, based on stored configurations, at least one activity that represents an incremental addition to a current activity level of the user; and
determining, based on the stored configurations, a statistical benefit of adding the at least one activity to the current activity level of the user.

20. The system of claim 1, wherein the generation of an integrative health-management insight comprises:
analyzing the integrative activity data over a period of time; and
identifying a noteworthy day over the period of time based on at least analyte levels of the user and activities of the user.

21. The system of claim 1, wherein the computer system is further operable to:
organize the integrative activity data into one or more user interface containers of related data; and
present the organized integrative activity data to the user.

22. The system of claim 1, wherein the computer system is further operable to:
aggregate multiple sets of integrative activity data;
organize the aggregated multiple sets of integrative activity data into user interface containers of related data; and
present the organized aggregated multiple sets of integrative activity data to the user.

23. The system of claim 1, wherein the computer system comprises a smartphone associated with the user.

24. The system of claim 1, wherein the computer system comprises a smartphone associated with the user and a remote system in communication with the smartphone.

25. The system of claim 1, wherein the analyte sensor system comprises a continuous glucose monitor, the first time series comprising glucose levels of the user.

26. The system of claim 1, wherein the activity information identifies a second activity of a second defined activity type, the second activity overlapping the activity of the defined activity type.

27. The system of claim 26, wherein the activity and the second activity share at least one of a start time or an end time.

28. A method of integrating health data from multiple wearables, the method comprising, by a system associated with a user:
receiving, from an analyte sensor system worn by the user, a first time series of analyte levels of the user;
receiving, from an activity monitor worn by the user, activity information that identifies an activity of a defined activity type;
correlating the analyte levels to the activity based on a time period of the activity; and
generating integrative activity data based on the correlation of the analyte levels to the activity.

29. The method of claim 28, wherein:
the first time series is received from the analyte sensor system on a continuous basis; and
the activity information is received in response to user-initiated synchronization of the activity monitor.

30. The method of claim 28, wherein the correlating is based on a span of time that includes at least one of a period of time before the time period of the activity or a period of time after the time period of the activity.

31. The method of claim 28, further comprising:
receiving a second time series from the activity monitor worn by the user; and
correlating the first time series from the analyte sensor system and the second time series from the activity monitor based on the time period of the activity, wherein the generated integrative activity data is based on the correlated first time series and second time series.

32. The method of claim 31, wherein:
the first time series comprises analyte levels of the user; and
the second time series comprises at least one of heart rates, steps, or global positioning system (GPS) data.

33. The method of claim 31, wherein the generating integrative activity data comprises generating a graphical control element that compares the first time series with the second time series over a span of time comprising at least a portion of the time period of the activity.

34. The method of claim 33, further comprising causing the graphical control element to be displayed.

35. The method of claim 34, wherein the graphical control element comprises a change option, the method further comprising:
receiving a user instruction, via the change option, to compare a third time series and a fourth time series over the span of time;
automatically generating a new graphical control element that compares the third time series with the fourth time series over the span of time; and
automatically causing the new graphical control element to be displayed.

36. The method of claim 35, wherein the third time series and the fourth time series are different from each of the first time series and the second time series.

37. The method of claim 34, wherein the graphical control element comprises an option to change a span of time over which the first time series and the second time series are compared, the method further comprising:
receiving a user instruction, via the change option, to change the span of time;
automatically generating a new graphical control element that compares the first time series with the second time series over a changed span of time; and
automatically causing the new graphical control element to be displayed.

38. The method of claim 37, wherein the changed span of time comprises at least one of time before the time period of the activity or time after the time period of the activity.

39. The method of claim 28, wherein the generating integrative activity data comprises identifying an analyte event during the time period of the activity based on the first time series.

40. The method of claim 39, wherein the analyte event relates to at least one of the analyte levels being below a threshold.

41. The method of claim 39, further comprising receiving a second time series from the activity monitor worn by the user, wherein the generating integrative activity data further comprises correlating the analyte event to at least one parameter from the second time series based on a time of the analyte event.

42. The method of claim 41, wherein the at least one parameter comprises at least one of heart rate or GPS data.

43. The method of claim 39, further comprising generating an integrative activity insight based on the integrative activity data, wherein the integrative activity data comprises a recommendation for future activities of the user.

44. The method of claim 39, wherein the generating an integrative activity insight comprises:
identifying a possible trigger of the analyte event based on stored configurations;
validating existence of the possible trigger relative to the analyte event; and
generating a recommendation related to the possible trigger responsive to the validating.

45. The method of claim 28, further comprising generating an integrative health-management insight based on the integrative activity data, the integrative health-management insight comprising an activity recommendation for the user.

46. The method of claim 45, wherein the generating an integrative health-management insight comprises:
analyzing the integrative activity data over a period of time;
identifying, based on stored configurations, at least one activity that represents an incremental addition to a current activity level of the user; and
determining, based on the stored configurations, a statistical benefit of adding the at least one activity to the current activity level of the user.

47. The method of claim 28, wherein the generating an integrative health-management insight comprises:
analyzing the integrative activity data over a period of time; and
identifying a noteworthy day over the period of time based on at least analyte levels of the user and activities of the user.

48. The method of claim 28, further comprising:
organizing the integrative activity data into one or more user interface containers of related data; and
presenting the organized integrative activity data to the user.

49. The method of claim 28, further comprising:
aggregating multiple sets of integrative activity data;
organizing the aggregated multiple sets of integrative activity data into user interface containers of related data; and
presenting the organized aggregated multiple sets of integrative activity data to the user.

50. The method of claim 28, wherein the system comprises a smartphone associated with the user.

51. The method of claim 28, wherein the system comprises a smartphone associated with the user and a remote system in communication with the smartphone.

52. The method of claim 28, wherein the analyte sensor system comprises a continuous glucose monitor, the first time series comprising glucose levels of the user.

53. The method of claim 28, wherein the activity information identifies a second activity of a second defined activity type, the second activity overlapping the activity of the defined activity type.

54. The method of claim 53, wherein the activity and the second activity share at least one of a start time or an end time.

55. A computer-program product comprising a non-transitory computer-usable medium having computer-readable program code embodied therein, the computer-readable program code adapted to be executed to implement a method comprising:
receiving, from an analyte sensor system worn by a user, a first time series of analyte levels of the user;
receiving, from an activity monitor worn by the user, activity information that identifies an activity of a defined activity type;
correlating the analyte levels to the activity based on a time period of the activity; and
generating integrative activity data based on the correlation of the analyte levels to the activity.
